(19) United States
(12) Patent Application Publication
(10) Pub. No.: US 2022/0185959 A1
(43) Pub. Date: Jun. 16, 2022

(54) COMPOUND, RESIN COMPOSITION AND POLYMERIZATION PRODUCT

(71) Applicant: TDK CORPORATION, Tokyo (JP)

(72) Inventor: Takashi INAGAKI, Tokyo (JP)

(73) Assignee: TDK CORPORATION, Tokyo (JP)

(21) Appl. No.: 17/439,905

(22) PCT Filed: Mar. 24, 2020
(86) PCT No.: PCT/JP2020/012909

§ 371 Date: Sep. 16, 2021

(30) Foreign Application Priority Data
Mar. 29, 2019 (JP) 2019-068679

(51) Int. Cl. C08G 65/42

(52) U.S. Cl. CPC C08G 65/42

(57) ABSTRACT

A compound having end groups each having a reactive group that are disposed at both ends respectively, and between the end groups, either or both of a first structure in which an aromatic cyclic group, an ether oxygen, a methylene group, an aromatic cyclic group, a methylene group, an ether oxygen and an aromatic cyclic group are bonded together in this order and a second structure in which an aromatic cyclic group, a methylene group, an ether oxygen, an aromatic cyclic group, an ether oxygen, a methylene group and an aromatic cyclic group are bonded together in this order.

### TECHNICAL FIELD

The present disclosure relates to a compound, a resin composition and a polymerization product.

Priority is claimed on Japanese Patent Application No. 2019-068679, filed in Japan on Mar. 29, 2019, the content of which is incorporated herein by reference.

### BACKGROUND ART

Recently, in association with demand for reduction in the size of electronic devices, treatment of heat that is generated from electronic components and the like has become important. As a method for improving the heat dissipation properties of electronic components, a method of using a resin material from which a polymer having a high thermal conductivity can be obtained as a material for electronic components is an exemplary example.

As a method for increasing the thermal conductivity of polymers, a method of using a resin material containing a highly thermally conductive filler is known. For example, Patent Document 1 discloses a thermally conductive material having a structure in which fine thermally conductive particles are dispersed in a polymer matrix.

In addition, as a method for increasing the thermal conductivity of polymers, the use of a liquid crystalline resin has been proposed. For example, Patent Document 2 discloses an insulating composition containing a liquid crystalline resin obtained by polymerizing a resin composition containing a monomer having a mesogenic group.

### CITATION LIST

### Patent Literature

### [Patent Document 1]

Japanese Unexamined Patent Application, First Publication No. 2010-65064

### [Patent Document 2]

Japanese Unexamined Patent Application, First Publication No. H11-323162

### SUMMARY OF THE INVENTION

### Technical Problem

However, when the thermal conductivity of a polymer is increased by increasing the amount of a filler in a resin material, the workability of the polymer deteriorates. Therefore, there have been cases where a polymer having a sufficiently high thermal conductivity cannot be obtained from conventional resin materials. In addition, with conventional resins, the thermal conductivity of polymers is insufficient, and there is a demand for a resin from which a polymer having a higher thermal conductivity can be obtained.

The present disclosure has been made in consideration of the above-described problem, and an objective of the present invention is to provide a compound from which a polymer having a high thermal conductivity can be obtained.

In addition, another objective of the present disclosure is to provide a resin composition containing the compound of the present disclosure and a polymerization product containing a polymer of the resin composition.

### Solution to Problem

In order to solve the above-described problem, regarding compounds that can be used as a raw material of resins, the present inventors paid attention to skeletons and end groups of the compounds and repeated intensive studies.

As a result, the present inventors found that a specific compound that has a structure in which an aromatic cyclic group that may have a substituent, an ether oxygen and a methylene group are bonded together in a specific order and that has end groups each having a reactive group that bond to both ends, respectively, is preferable.

That is, the present disclosure relates to the following inventions.

(1) A compound having

end groups each having a reactive group that are disposed at both ends respectively, and

between the end groups, either or both of:

a first structure in which an aromatic cyclic group, an ether oxygen, a methylene group, an aromatic cyclic group, a methylene group, an ether oxygen and an aromatic cyclic group are bonded together in this order, and

a second structure in which an aromatic cyclic group, a methylene group, an ether oxygen, an aromatic cyclic group, an ether oxygen, a methylene group and an aromatic cyclic group are bonded together in this order.

[2] The compound according to [1], including

a first aromatic cyclic unit composed of a first aromatic cyclic group and two ether oxygens bonding to the first aromatic cyclic group,

a second aromatic cyclic unit composed of a second aromatic cyclic group and two methylene groups bonding to the second aromatic cyclic group and

a third aromatic cyclic unit composed of a third aromatic cyclic group and an end group having a reactive group that bonds to the third aromatic cyclic group,

in which the compound includes a skeleton in which the first aromatic cyclic units and the second aromatic cyclic units are alternately disposed, and

the first aromatic cyclic units are disposed at both ends of the skeleton and bonded to the third aromatic cyclic groups via methylene groups, or the second aromatic cyclic units are disposed at both ends of the skeleton and bonded to the third aromatic cyclic groups via ether oxygens.

[3] The compound according to [1] that is represented by General Formula (1) below or General Formula (2) below.

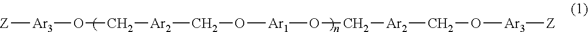

(In Formula (1), Ar₁ each independently represents a first aromatic cyclic group that may have a substituent, Are each independently represents a second aromatic cyclic group that may have a substituent, and Ar₃ each independently represents a third aromatic cyclic group that may have a substituent; Z each independently represents an end group having a reactive group; and n is an integer of 0 or larger.)

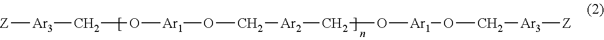

(In Formula (2), Ar₁ each independently represents a first aromatic cyclic group that may have a substituent, Are each independently represents a second aromatic cyclic group that may have a substituent, and Ar₃ each independently represents a third aromatic cyclic group that may have a substituent; Z each independently represents an end group having a reactive group. n is an integer of 0 or larger.)

[4] The compound according to [2] or [3], in which any one or more of the first aromatic cyclic group, the second aromatic cyclic group and the third aromatic cyclic group are any of aromatic cyclic groups represented by General Formulae (3) to (7) below.

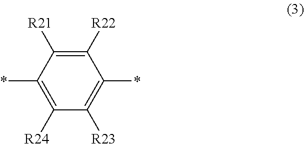

(In Formula (3), R21 to R24 are each independently any one selected from the group consisting of hydrogen, a methyl group, a trifluoromethyl group, a halogen group and a nitro group.)

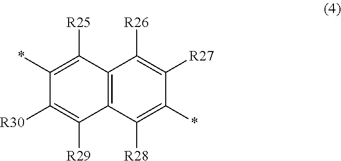

(In Formula (4), R25 to R30 are each independently any one selected from the group consisting of hydrogen, a methyl group, a trifluoromethyl group, a halogen group and a nitro group.)

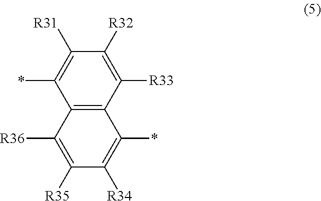

(In Formula (5), R31 to R36 are each independently any one selected from the group consisting of hydrogen, a methyl group, a trifluoromethyl group, a halogen group and a nitro group.)

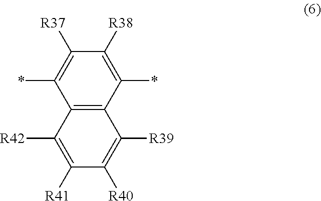

(In Formula (6), R37 to R42 are each independently any one selected from the group consisting of hydrogen, a methyl group, a trifluoromethyl group, a halogen group and a nitro group.)

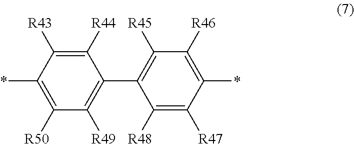

(In Formula (7), R43 to R50 are each independently any one selected from the group consisting of hydrogen, a methyl group, a trifluoromethyl group, a halogen group and a nitro group.)

[5] The compound according to any one of [2] to [4], in which any one or more of the first aromatic cyclic group, the second aromatic cyclic group and the third aromatic cyclic group are a para-phenylene group that may have a substituent.

[6] The compound according to any one of [2] to [5], in which the second aromatic cyclic group is a para-phenylene group.

[7] The compound according to any one of [1] to [6] that is represented by General Formula (8) or General Formula (9) below.

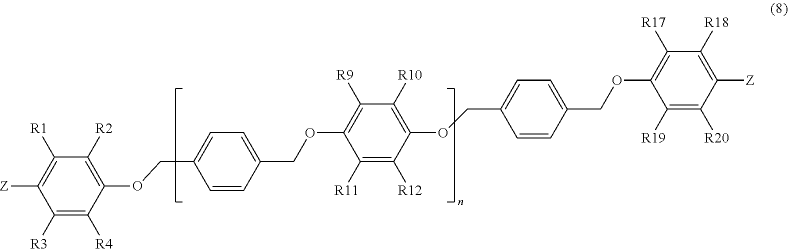

(In Formula (8), R1 to R4, R9 to R12 and R17 to R20 are each independently any one selected from the group consisting of hydrogen, a methyl group, a trifluoromethyl group, a halogen group and a nitro group; Z each independently represents an end group having a reactive group; and n is an integer of 0 or larger.)

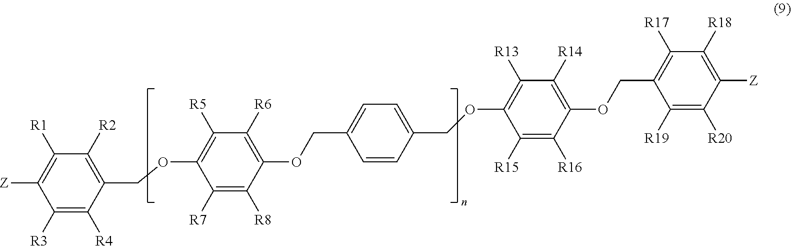

(In Formula (9), R1 to R8 and R13 to R20 are each independently any one selected from the group consisting of hydrogen, a methyl group, a trifluoromethyl group, a halogen group and a nitro group; Z each independently represents an end group having a reactive group; and n is an integer of 0 or larger.)

[8] The compound according to any one of [1] to [7], in which the end group having a reactive group is —OH, —COOR (R is an alkyl group), —NH₂, —COOH, —COCl, —CH═CH₂, —CH₂OH, —O—COR (R is an alkyl group) or any of end groups represented by Formulae (10) to (12) below.

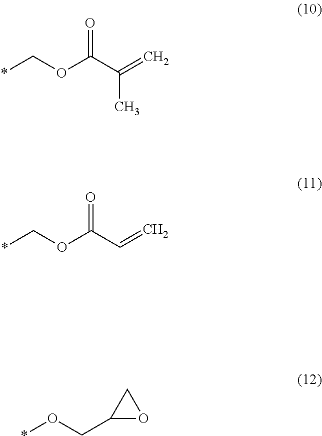

[9] A resin composition containing the compound according to any one of [1] to [8]. [10] A polymerization product containing a polymer of the resin composition according to [9].

### Advantageous Effects of Invention

The compound of the present disclosure has, between the end groups each having a reactive group that are disposed at both ends respectively, either or both of the first structure in which an aromatic cyclic group, an ether oxygen, a methylene group, an aromatic cyclic group, a methylene group, an ether oxygen and an aromatic cyclic group are bonded together in this order and/or the second structure in which an aromatic cyclic group, a methylene group, an ether oxygen, an aromatic cyclic group, an ether oxygen, a methylene group and an aromatic cyclic group are bonded together in this order. The first structure and the second structure each have a structure in which aromatic cyclic groups that are each a mesogenic group developing liquid crystallinity and impart rigidity, methylene groups and ether oxygens that impart mobility are disposed in a specific order. Due to this fact, the compound of the present disclosure is capable of stabilizing a smectic liquid crystal phase with appropriate mobility intrinsic to the mesogenic groups in spite of having no long side chains which are typically observable in liquid crystal molecules. Therefore, the compound of the present disclosure has high orientation, and a polymerization product which has a smectic liquid crystal structure and is highly thermally conductive due to suppression of the scattering of phonons can be obtained by polymerizing the compound of the present disclosure.

### DESCRIPTION OF EMBODIMENTS

Hereinafter, preferable examples of the present disclosure will be described in detail.

### “Compound”

A compound of the present embodiment has a first structure and/or a second structure between end groups each having a reactive group that are disposed at both ends respectively.

The first structure is a structure in which an aromatic cyclic group, an ether oxygen, a methylene group, an aromatic cyclic group, a methylene group, an ether oxygen and an aromatic cyclic group are bonded together in this order.

The second structure is a structure in which an aromatic cyclic group, a methylene group, an ether oxygen, an aromatic cyclic group, an ether oxygen, a methylene group and an aromatic cyclic group are bonded together in this order.

The compound of the present embodiment preferably includes a first aromatic cyclic unit, a second aromatic cyclic unit and a third aromatic cyclic unit, all of which will be described below.

The first aromatic cyclic unit is composed of a first aromatic cyclic group and two ether oxygens bonding to the first aromatic cyclic group.

The second aromatic cyclic unit is composed of a second aromatic cyclic group and two methylene groups bonding to the second aromatic cyclic group.

The third aromatic cyclic unit is composed of a third aromatic cyclic group and an end group having a reactive group that bonds to the third aromatic cyclic group.

The compound of the present embodiment preferably includes a skeleton in which the first aromatic cyclic units and the second aromatic cyclic units are alternately disposed once or more.

At both ends of the skeleton, the first aromatic cyclic units may be disposed or the second aromatic cyclic units may be disposed. When the first aromatic cyclic units or the second aromatic cyclic units are disposed at both ends of the skeleton, the skeleton is preferably provided with a symmetric structure.

In the compound of the present embodiment, in a case where the first aromatic cyclic units are disposed at both ends of the skeleton, the first aromatic cyclic units are bonded to the third aromatic cyclic groups with the methylene groups.

In addition, in the compound of the present embodiment, in a case where the second aromatic cyclic units are disposed at both ends of the skeleton, the second aromatic cyclic units are bonded to the third aromatic cyclic groups with the ether oxygens.

All of the first aromatic cyclic group, the second aromatic cyclic group and the third aromatic cyclic group in the compound of the present embodiment may be an aromatic cyclic group and may have a substituent. The expression “the aromatic cyclic group may have a substituent” may mean that the aromatic cyclic group has a substituent or has no substituent. The first aromatic cyclic group, the second aromatic cyclic group and the third aromatic cyclic group may be different from one another and may be partially or entirely identical to one another, which can be appropriately determined depending on the application or the like of the compound.

In a case where the compound of the present embodiment has a plurality of the first aromatic cyclic groups, the plurality of first aromatic cyclic groups may be different from each other or may be partially or entirely identical to each other. The plurality of first aromatic cyclic groups is preferably identical to each other since the compound in which the plurality of first aromatic cyclic groups is all identical to each other can be easily produced.

In addition, in a case where the compound of the present embodiment has a plurality of the second aromatic cyclic groups, the plurality of second aromatic cyclic groups may be different from each other or may be partially or entirely identical to each other. The plurality of second aromatic cyclic groups is preferably identical to each other since the compound in which the plurality of second aromatic cyclic groups is all identical to each other can be easily produced.

In addition, the third aromatic cyclic groups that are disposed at both ends of the skeleton of the compound of the present embodiment may be different from each other or identical to each other. The third aromatic cyclic groups are preferably identical to each other since the compound in which the third aromatic cyclic groups that are disposed at both ends of the skeleton are identical to each other can be easily produced.

In the compound of the present embodiment, the substituents in the first aromatic cyclic group, the second aromatic cyclic group and the third aromatic cyclic group are preferably any one selected from the group consisting of a methyl group, a trifluoromethyl group, a halogen group and a nitro group, can be appropriately determined depending on the application or the like of the compound, and are not particularly limited. Among these substituents, particularly, a methyl group, a trifluoromethyl group and a halogen group are preferable from the viewpoint of chemical stability and the reduction of environmental burden and a methyl group is particularly preferable.

Any one or more of the first aromatic cyclic group, the second aromatic cyclic group and the third aromatic cyclic group in the compound of the present embodiment may be any of aromatic cyclic groups represented by General Formulae (3) to (7) below. In a case where any one or more of the first aromatic cyclic group, the second aromatic cyclic group and the third aromatic cyclic group are any of groups represented by General Formulae (3) to (7), a polymer having a higher thermal conductivity can be obtained and, furthermore, the handleability of the polymer becomes favorable, which is preferable.

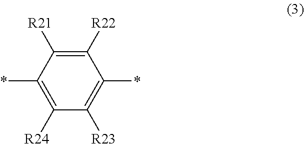

(In Formula (3), R21 to R24 are each independently any one selected from the group consisting of hydrogen, a methyl group, a trifluoromethyl group, a halogen group and a nitro group.)

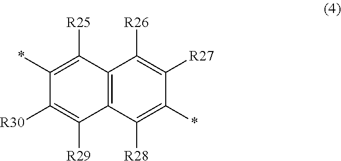

(In Formula (4), R25 to R30 are each independently any one selected from the group consisting of hydrogen, a methyl group, a trifluoromethyl group, a halogen group and a nitro group.)

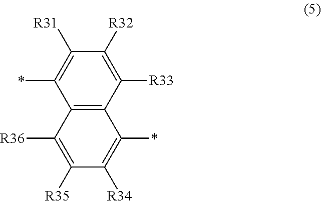

(In Formula (5), R31 to R36 are each independently any one selected from the group consisting of hydrogen, a methyl group, a trifluoromethyl group, a halogen group and a nitro group.)

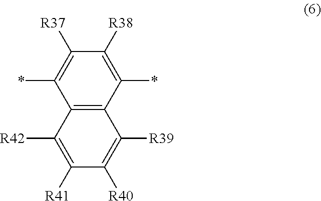

(In Formula (6), R37 to R42 are each independently any one selected from the group consisting of hydrogen, a methyl group, a trifluoromethyl group, a halogen group and a nitro group.)

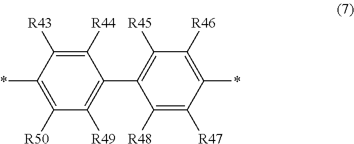

(In Formula (7), R43 to R50 are each independently any one selected from the group consisting of hydrogen, a methyl group, a trifluoromethyl group, a halogen group and a nitro group.)

Any one or more of the first aromatic cyclic group, the second aromatic cyclic group and the third aromatic cyclic group in the compound of the present embodiment are preferably a phenylene group that may have a substituent in order to make a compound from which a polymer having a higher thermal conductivity can be obtained. The phenylene group of the phenylene group that may have a substituent may be any one of an ortho-phenylene group, a meta-phenylene group and a para-phenylene group. The phenylene group is particularly preferably a para-phenylene group since the compound then has a skeleton exhibiting high orientation.

In the compound of the present embodiment, particularly, the second aromatic cyclic group is preferably a para-phenylene group. In such a case, the compound has a skeleton including a structure in which the methylene groups bond to both sides of the para-phenylene group and thus exhibits higher orientation. As a result, a polymer having an even higher thermal conductivity can be obtained from the compound. In addition, when the second aromatic cyclic group is a para-phenylene group having no substituent, procurement of a raw material is easy, and the compound becomes favorable in solubility in solvents at low melting points.

Examples of the compound of the present embodiment include compounds represented by General Formula (1) below or General Formula (2) below.

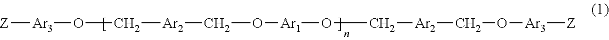

(In Formula (1), Ar₁ each independently represents the first aromatic cyclic group that may have a substituent, Ar₂ each independently represents the second aromatic cyclic group that may have a substituent, and Ar₃ each independently represents the third aromatic cyclic group that may have a substituent; Z each independently represents an end group having a reactive group; n is an integer of 0 or larger.)

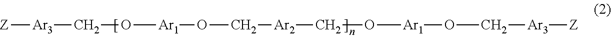

(In Formula (2), Ar₁ each independently represents the first aromatic cyclic group that may have a substituent, Ar₂ each independently represents the second aromatic cyclic group that may have a substituent, and Ar₃ each independently represents the third aromatic cyclic group that may have a substituent; Z each independently represents an end group having a reactive group; n is an integer of 0 or larger.)

The compounds represented by General Formula (1) and General Formula (2) include the first aromatic cyclic unit (indicated by —O—Ar₁—O— in Formula (1) and Formula (2)), the second aromatic cyclic unit (indicated by —CH₂—Ar₁—CH₂— in Formula (1)) and the third aromatic cyclic unit (indicated by —Ar₃—Z in Formula (1) and Formula (2)).

In the compounds represented by General Formula (1) and General Formula (2), the first aromatic cyclic unit has the first aromatic cyclic group (indicated by Ar₁ in Formula (1) and Formula (2)) and two ether oxygens bonding to the first aromatic cyclic group.

The second aromatic cyclic unit has the second aromatic cyclic group (indicated by Are in Formula (1) and Formula (2)) and two methylene groups bonding to the second aromatic cyclic group.

The third aromatic cyclic unit is composed of the third aromatic cyclic group (indicated by Ar₃ in Formula (1) and Formula (2)) and an end group having a reactive group that bonds to the third aromatic cyclic group (indicated by Z in Formula (1) and Formula (2)).

The compound represented by General Formula (1) includes a skeleton in which the first aromatic cyclic units and the second aromatic cyclic units are alternately disposed in a chain shape and has a skeleton in which both ends are terminated with the second aromatic cyclic units. In the compound represented by General Formula (1), the methylene groups in the second aromatic cyclic unit are disposed at both ends of the skeleton, and the second aromatic cyclic unit is bonded to the third aromatic cyclic group indicated by Ar₃ in Formula (1) with the ether oxygen.

In addition, the compound represented by General Formula (2) includes a skeleton in which the first aromatic cyclic units and the second aromatic cyclic units are alternately disposed in a chain shape and has a skeleton in which both ends are terminated with the first aromatic cyclic units. In the compound represented by General Formula (2), the ether oxygens in the first aromatic cyclic unit are disposed at both ends of the skeleton, and the first aromatic cyclic unit is bonded to the third aromatic cyclic group indicated by Ar₃ in Formula (2) with the methylene group.

Therefore, both ends of the compounds represented by General Formulae (1) and (2) are the end groups having a reactive group, which are indicated by Z in Formula (1) and Formula (2), that bond to the third aromatic cyclic group.

Examples of a compound in which all of the first aromatic cyclic group, the second aromatic cyclic group and the third aromatic cyclic group are a para-phenylene group that may have a substituent, which is represented by General Formula (3), in the compound of the present embodiment include compounds represented by General Formula (13) below or General Formula (14) below.

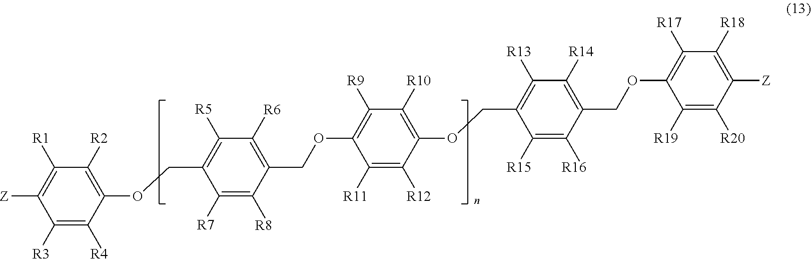

(In Formula (13), R1 to R20 are each independently any one selected from the group consisting of hydrogen, a methyl group, a trifluoromethyl group, a halogen group and a nitro group. Z each independently represents an end group having a reactive group. n is an integer of 0 or larger.)

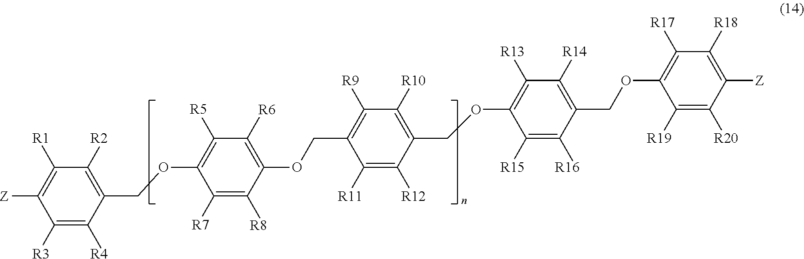

(In Formula (14), R1 to R20 are each independently any one selected from the group consisting of hydrogen, a methyl group, a trifluoromethyl group, a halogen group and a nitro group. Z each independently represents an end group having a reactive group. n is an integer of 0 or larger.)

The compounds represented by General Formula (13) and General Formula (14) have the first aromatic cyclic unit composed of a para-phenylene group that may have a substituent as the first aromatic cyclic group and two ether oxygens disposed at para positions with respect to the first aromatic cyclic group. In addition, the compounds have the second aromatic cyclic unit composed of a para-phenylene group that may have a substituent as the second aromatic cyclic group and two methylene groups disposed at para positions with respect to the first aromatic cyclic group. Furthermore, the compounds have the third aromatic cyclic unit composed of a para-phenylene group that may have a substituent as the third aromatic cyclic group and end groups having a reactive group (indicated by Z in Formulae (13) and (14)).

The compound represented by General Formula (13) has a skeleton in which the first aromatic cyclic units and the second aromatic cyclic units are alternately disposed and both ends are terminated with the second aromatic cyclic units. Furthermore, the end group having a reactive group and the ether oxygen bonded to the skeleton are disposed at para positions with respect to the para-phenylene group that may have a substituent as the third aromatic cyclic group, and the third aromatic cyclic units are disposed symmetrically with respect to the skeleton. Due to these facts, the skeleton of the compound represented by General Formula (13) exhibits liquid crystallinity and exhibits high orientation. Therefore, a polymer having a more favorable thermal conductive property can be obtained from the compound represented by General Formula (13).

In addition, the compound represented by General Formula (14) has a skeleton in which the first aromatic cyclic units and the second aromatic cyclic units are alternately disposed and both ends are terminated with the first aromatic cyclic units. Furthermore, the end group having a reactive group and the methylene group bonded to the skeleton are disposed at para positions with respect to the para-phenylene group that may have a substituent as the third aromatic cyclic group, and the third aromatic cyclic units are disposed symmetrically with respect to the skeleton. Due to these facts, the skeleton of the compound represented by General Formula (14) exhibits liquid crystallinity and exhibits high orientation. Therefore, a polymer having a more favorable thermal conductive property can be obtained from the compound represented by General Formula (14).

Examples of a compound in which the first aromatic cyclic group and the third aromatic cyclic group are the para-phenylene group that may have a substituent, which is represented by Formula (3), and the second aromatic cyclic group is the para-phenylene group in the compound of the present embodiment include compounds represented by General Formula (8) below or General Formula (9) below.

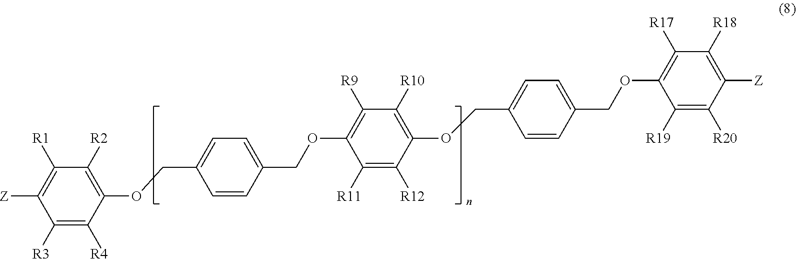

(In Formula (8), R1 to R4, R9 to R12 and R17 to R20 are each independently any one selected from the group consisting of hydrogen, a methyl group, a trifluoromethyl group, a halogen group and a nitro group. Z each independently represents an end group having a reactive group. n is an integer of 0 or larger.)

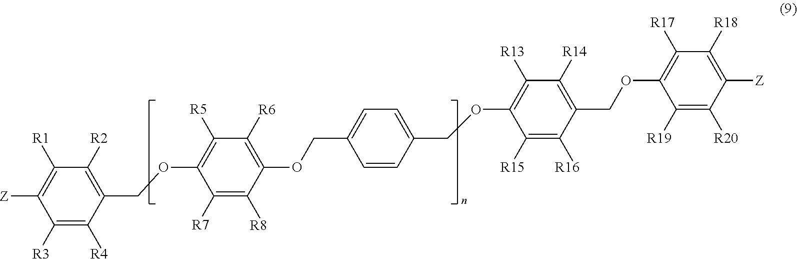

(In Formula (9), R1 to R8 and R13 to R20 are each independently any one selected from the group consisting of hydrogen, a methyl group, a trifluoromethyl group, a halogen group and a nitro group. Z each independently represents an end group having a reactive group. n is an integer of 0 or larger.)

In the compounds represented by General Formula (8) and General Formula (9), the first aromatic cyclic group and the third aromatic cyclic group are the para-phenylene group that may have a substituent, which is represented by General Formula (3), and the second aromatic cyclic group is a para-phenylene group. Therefore, the compounds represented by General Formula (8) and General Formula (9) have a skeleton including a structure in which the methylene groups bond to both sides of the para-phenylene group and exhibit higher orientation. Therefore, according to the compounds represented by General Formula (8) and General Formula (9), a polymer having a more favorable thermal conductive property can be obtained. In addition, in the compounds represented by General Formula (8) and General Formula (9), since the second aromatic cyclic group is a para-phenylene group having no substituent, procurement of a raw material is easy.

In the compound represented by General Formula (8), the end group having a reactive group and the ether oxygen bonded to the skeleton are disposed at para positions with respect to the para-phenylene group that may have a substituent as the third aromatic cyclic group.

In the compound represented by General Formula (9), the end group having a reactive group and the methylene group bonded to the skeleton are disposed at para positions with respect to the para-phenylene group that may have a substituent as the third aromatic cyclic group.

In the compound represented by General Formula (8), since the third aromatic cyclic group and the ether oxygen bonded to the skeleton are bonded to each other, compared with the compound represented by General Formula (9) in which the third aromatic cyclic group and the methylene group bonded to the skeleton are bonded to each other, a bonding portion between the end group having a reactive group and the skeleton does not become too rigid, and the balance between orientation and molecular mobility becomes favorable. As a result, the compound represented by General Formula (8) has sufficient solubility in solvents, and a polymer having a favorable thermal conductive property can be obtained from the compound.

In the compounds represented by General Formula (8) and General Formula (9), the first aromatic cyclic group and the third aromatic cyclic group are preferably a para-phenylene group having one methyl group. In this case, compared with a case where all of the first aromatic cyclic group, the second aromatic cyclic group and the third aromatic cyclic group are a para-phenylene group having no substituent, crystallinity in the skeleton deteriorates, and a smectic liquid crystal phase is stabilized. As a result, a polymer having a favorable thermal conductive property can be obtained from the compound.

In the compound of the present embodiment, the first aromatic cyclic group and the third aromatic cyclic group are preferably identical to each other. When the first aromatic cyclic group and the third aromatic cyclic group are identical to each other, compared with a case where the first aromatic cyclic group and the third aromatic cyclic group are different from each other, the compound can be easily produced and becomes excellent in terms of productivity. Particularly, in a case where the first aromatic cyclic group and the third aromatic cyclic group are identical to each other and the second aromatic cyclic group is a para-phenylene group, the compound can be easily produced and becomes excellent in terms of productivity.

In an epoxy resin of the present embodiment, the first aromatic cyclic group and the second aromatic cyclic group may be identical to each other or may be different from each other. Both the first aromatic cyclic group and the second aromatic cyclic group may be a para-phenylene group having no substituent. In this case, procurement of a raw material is easy, which is preferable. In addition, in a case where the first aromatic cyclic group and the second aromatic cyclic group are different from each other, compared with a case where the first aromatic cyclic group and the second aromatic cyclic group are identical to each other, the symmetry of the structure in the skeleton becomes poor. Therefore, the crystallinity of the compound deteriorates, and a smectic liquid crystal phase is stabilized. As a result, a polymer having a favorable thermal conductive property can be obtained from the compound.

In the compounds represented by General Formulae (1), (2), (8), (9), (13) and (14), n is the number of repeating units written in a parenthesis. In the compounds represented by General Formulae (1), (2), (8), (9), (13) and (14), n is an integer of zero or larger. n is zero or larger such that an effect of having the above-described skeleton that improves the thermal conductivity of polymers can be obtained and n is preferably one or larger and more preferably two or larger such that the effect of having the above-described skeleton that improves the thermal conductivity of polymers becomes more significant. n may be three or larger, five or larger, eight or larger, 10 or larger or 12 or larger as necessary. In addition, the upper limit of n in General Formulae (1), (2), (8), (9), (13) and (14) is not particularly limited, but is preferably 20 or smaller in order to ensure the solubility of the compound in solvents and may be 16 or smaller, 14 or smaller or 12 or smaller. In order for the epoxy resin to become more favorable in solubility in solvents, the upper limit is more preferably 10 or smaller and still more preferably six or smaller.

As described above, n can be selected as necessary. n may be an even number or an odd number. For example, n may be one or more of integers indicated by 0, 1, 2, 3, 4, 5, 6, 7, 8, 9, 10, 11, 12, 13, 14, 15, 16, 17, 18, 19 and 20. For example, the lower limit of n may be any of integers within a range of 0 to 20, and the upper limit of n may be any of integers within a range of 0 to 20. Specifically, for example, n may be an integer within a range of 0 to 20, an integer within a range of 0 to 15, an integer within a range of 0 to 10, an integer within a range of 0 to 8, an integer within a range of 0 to 5, an integer within a range of 0 to 2 or an integer within a range of 1 to 2.

The skeleton of the compound of the present embodiment has a repeating unit composed of one first aromatic cyclic unit and one second aromatic cyclic unit. The compound of the present embodiment may be a mixture including a plurality of kinds of compounds having different numbers of repeating units or may be a single compound having the same number of repeating units.

In a case where the compound of the present embodiment is a mixture including a plurality of kinds of compounds having different numbers of repeating units, the average polymerization degree, which is the average value of the numbers of repeating units of the compounds in the mixture, can be optionally selected, but is preferably 1.0 to 6.0, more preferably 1.5 to 5.5 and still more preferably 2.0 to 5.0. The average polymerization degree may be 1.0 to 4.0, 3.0 to 4.0 or the like as necessary. When the average polymerization degree is 1.0 or higher, a polymer having an even higher thermal conductivity can be obtained from the compound. In addition, when the average polymerization degree is 6.0 or lower, the compound becomes more favorable in solubility in solvents.

The compound of the present embodiment has the first structure or the second structure between end groups each having a reactive group that are disposed at both ends respectively, even when n in the compounds represented by General Formulae (1), (2), (8), (9), (13) and (14) is zero. The first structure is a structure in which an aromatic cyclic group, an ether oxygen, a methylene group, an aromatic cyclic group, a methylene group, an ether oxygen and an aromatic cyclic group are bonded together in this order. The second structure is a structure in which an aromatic cyclic group, a methylene group, an ether oxygen, an aromatic cyclic group, an ether oxygen, a methylene group and an aromatic cyclic group are bonded together in this order. The first structure and the second structure each have a structure in which aromatic cyclic groups that are each a mesogenic group developing liquid crystallinity and impart rigidity, and methylene groups and ether oxygens that impart mobility are disposed in a specific order. Due to this fact, according to the compound of the present embodiment, a polymerization product having a high thermal conductive property can be obtained.

In the compound of the present embodiment, since the end group having a reactive group is a group that easily bonds to the skeleton of the compound and a compound having a more favorable thermal conductive property can be obtained, the end group is preferably —OH, —COOR (R is an alkyl group), —NH₂, —COOH, —COCl, —CH═CH₂, —CH₂OH, —O—COR (R is an alkyl group) or any of end groups represented by Formulae (10) to (12) below and can be appropriately determined depending on the application or the like of the compound.

In a case where the end group having a reactive group is —COOR (R is an alkyl group), examples of R include a methyl group, an ethyl group, an n-propyl group, an i-propyl group and the like.

In addition, in a case where the end group having a reactive group is —O—COR (R is an alkyl group), examples of R include a methyl group, an ethyl group, an n-propyl group, an i-propyl group and the like. The compound in which the end group having a reactive group is —O—COR (R is an alkyl group) is polymerized by a decarboxylation reaction. Therefore, in a case where the end group having a reactive group is —O—COR (R is an alkyl group), R is preferably an alkyl group having a small molecular weight and most preferably a methyl group.

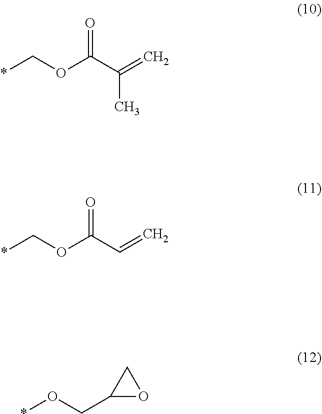

Specific examples of a preferable compound of the present embodiment include compounds represented by General Formula (A) to General Formula (C) and the like.

In the compound indicated by General Formula (A), the first aromatic cyclic group and the third aromatic cyclic group are a para-phenylene group having a methyl group, the second aromatic cyclic group is a para-phenylene group, the end group having a reactive group is an acetyloxy group (—O—COCH₃) and the acetyloxy group and an ether oxygen bonded to the skeleton are disposed at para positions with respect to the para-phenylene group having a methyl group as the third aromatic cyclic group.

In the compound indicated by General Formula (B), the first aromatic cyclic group is a para-phenylene group having a methyl group, the second aromatic cyclic group and the third aromatic cyclic group are a para-phenylene group, the end group having a reactive group is the end group indicated by Formula (10) and the end group having a reactive group and a methylene group bonded to the skeleton are disposed at para positions with respect to the para-phenylene group as the third aromatic cyclic group.

In the compound indicated by General Formula (C), the first aromatic cyclic group and the third aromatic cyclic group are a para-phenylene group having a methyl group, the second aromatic cyclic group is a para-phenylene group, the end group having a reactive group is the end group indicated by Formula (12) and the end group having a reactive group and an ether oxygen bonded to the skeleton are disposed at para positions with respect to the para-phenylene group having a methyl group as the third aromatic cyclic group.

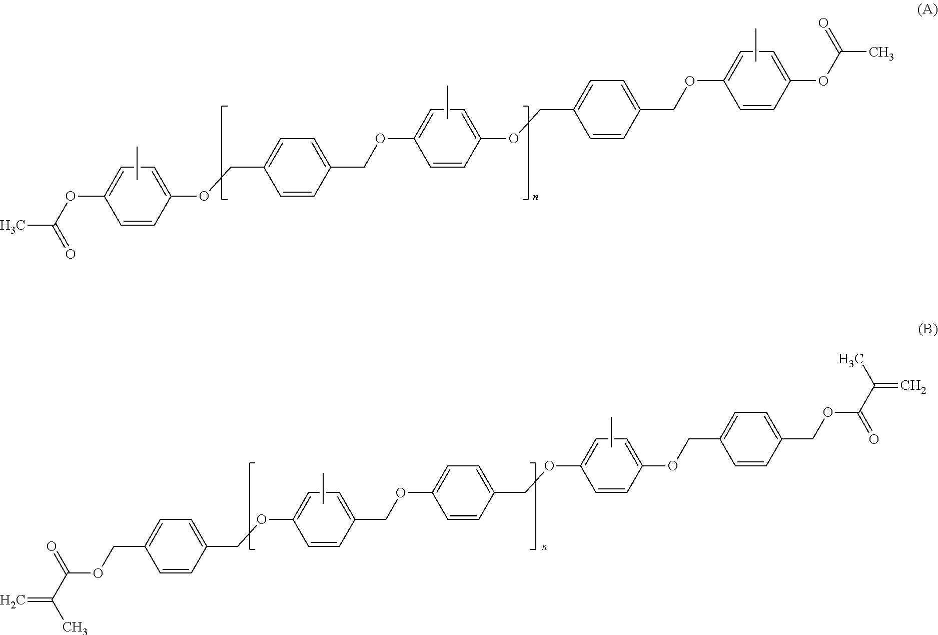

(In Formula (A), n is an integer of 0 or larger.)

(In Formula (B), n is an integer of 0 or larger.)

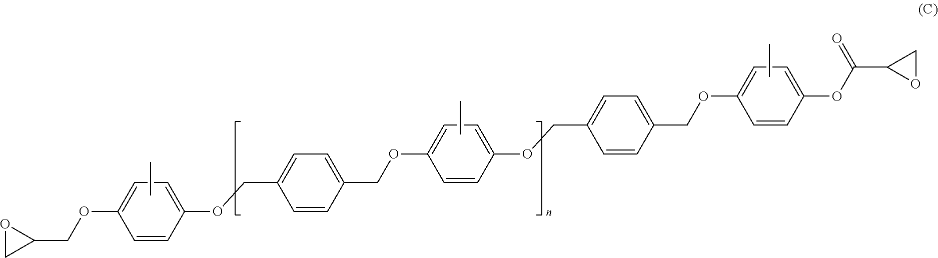

(In Formula (C), n is an integer of 0 or larger.)

### “Method for Producing Compound”

The compound of the present embodiment can be produced by, for example, a method described below.

A first raw material that is an aromatic compound having two phenolic hydroxyl groups and a second raw material that is an aromatic compound having a monohalogenated methyl group are prepared.

In addition, a bimolecular nucleophilic substitution reaction (S_{N}2 reaction) between the first raw material and the second raw material is caused to synthesize a first precursor compound having a structure which forms the skeleton in the compound of the present embodiment. The conditions for the reaction between the first raw material and the second raw material can be appropriately determined depending on the combination of the first raw material and the second raw material and are not particularly limited.

The first raw material that is used in the method for producing the compound of the present embodiment is an aromatic compound having two phenolic hydroxyl groups and is appropriately selected depending on the structure of a compound to be produced. Examples of the first raw material include methylhydroquinone, hydroquinone, tetramethylhydroquinone, trimethylhydroquinone, 2-(trifluoromethyl)-1,4-benzenediol, fluorohydroquinone, chlorohydroquinone, bromohydroquinone, 2,5-dihydroxynitrobenzene, tetrafluorohydroquinone, tetrachlorohydroquinone, tetrabromohydroquinone, 2,6-dihydroxynaphthalene, 1,5-dihydroxynaphthalene, 4,4′-dihydroxybiphenyl, 3,3′, 5,5′-tetramethylbiphenyl-4,4′-diol and the like.

The second raw material that is used in the method for producing the compound of the present embodiment is an aromatic compound having a monohalogenated methyl group and is appropriately selected depending on the structure of a compound to be produced. Examples of the second raw material include α,α′-dichloro-p-xylene, 1,4-bis(chloromethyl)-2-methylbenzene, 3,6-bis(chloromethyl)durene, 1,4-bis(bromomethyl)-2-fluorobenzene, 1,4-bis(bromomethyl)-2-chlorobenzene, 2-bromo-1,4-bis(bromomethyl)benzene, 1,4-bis(chloromethyl)-2-nitrobenzene, 1,4-bis(bromomethyl)-2,3,5,6-tetrafluorobenzene, α,α′,2,3,5,6-hexachloro-p-xylene, 1,2,4,5-tetrabromo-3,6-bis(bromomethyl)benzene, 1,2-dibromo-3,6-bis(chloromethyl)-4,5-dimethylbenzene, 1,4-bis(bromomethyl)-2,5-dimethylbenzene, 4,4′-bis(chloromethyl) biphenyl, 2,6-bis(bromomethyl)naphthalene, 1,5-bis(chloromethyl)naphthalene and the like.

Next, the obtained first precursor compound and a third raw material are reacted with each other to synthesize a second precursor compound. The conditions for the reaction between the first precursor compound and the third raw material can be appropriately determined depending on the combination of the first precursor compound and the third raw material and are not particularly limited.

The third raw material that is used in the method for producing the compound of the present embodiment is appropriately selected depending on the structure of an end group having a reactive group, the structure of a third aromatic cyclic group and the like in a compound to be produced. In addition, as the third raw material, different raw materials are used in a case where elements that are disposed at both ends of the skeleton of the previously-synthesized first precursor compound have a structure derived from the first raw material and a case where the elements have a structure derived from the second raw material, respectively.

In a case where the elements disposed at both ends of the skeleton of the first precursor compound have a structure derived from the first raw material, as the third raw material, similar to the second raw material, an aromatic compound having a monohalogenated methyl group is used. Specific examples thereof include α,α′-dichloro-p-xylene, 1,4-bis(chloromethyl)-2-methylbenzene, 3,6-bis(chloromethyl)durene, 1,4-bis(bromomethyl)-2-fluorobenzene, 1,4-bis(bromomethyl)-2-chlorobenzene, 2-bromo-1,4-bis(bromomethyl)benzene, 1,4-bis(chloromethyl)-2-nitrobenzene, 1,4-bis(bromomethyl)-2,3,5,6-tetrafluorobenzene, α,α′,2,3,5,6-hexachloro-p-xylene, 1,2,4,5-tetrabromo-3,6-bis(bromomethyl)benzene, 1,2-dibromo-3,6-bis(chloromethyl)-4,5-dimethylbenzene, 1,4-bis(bromomethyl)-2,5-dimethylbenzene, 4,4′-bis(chloromethyl) biphenyl, 2,6-bis(bromomethyl)naphthalene, 1,5-bis(chloromethyl)naphthalene and the like.

In a case where the elements disposed at both ends of the skeleton of the first precursor compound have a structure derived from the second raw material, as the third raw material, similar to the first raw material, an aromatic compound having two phenolic hydroxyl groups can be used. In addition, as the third raw material, an aromatic compound having one phenolic hydroxyl group and an amino group or a carboxyalkyl group may also be used. Specific examples thereof include methylhydroquinone, hydroquinone, tetramethylhydroquinone, trimethylhydroquinone, 2-(trifluoromethyl)-1,4-benzenediol, fluorohydroquinone, chlorohydroquinone, bromohydroquinone, 2,5-dihydroxynitrobenzene, tetrafluorohydroquinone, tetrachlorohydroquinone, tetrabromohydroquinone, 2,6-dihydroxynaphthalene, 1,5-dihydroxynaphthalene, 4,4′-dihydroxybiphenyl, 3,3′, 5,5′-tetramethylbiphenyl-4,4′-diol, p-aminophenol, 4-amino-m-cresol, methyl 4-hydroxybenzoate and the like.

Next, the second precursor compound obtained by the reaction between the first precursor compound and the third raw material and a compound having a structure from which the end group having a reactive group is to be derived are reacted with each other to obtain the compound of the present embodiment.

As a method for reacting the second precursor compound and the compound having a structure from which the end group having a reactive group is to be derived, a well-known method can be used, and there is no particular limitation.

In the case of producing a compound having a structure in which the first aromatic cyclic group and the third aromatic cyclic group are identical to each other or a compound having a structure in which the second aromatic cyclic group and the third aromatic cyclic group are identical to each other as the compound, there are cases where the step of reacting the first precursor compound and the third raw material is skipped.

In a case where the end group having a reactive group in the compound of the present embodiment is —OH, the compound can be produced by, for example, a method in which the first raw material is used in a larger substance amount (number of moles) than the second raw material in a step of producing the first precursor compound. In this case, the first precursor compound becomes the compound of the present embodiment.

In a case where the end group having a reactive group in the compound of the present embodiment is —COOR (R is an alkyl group), the first precursor compound having a monohalogenated methyl group is produced by, for example, using the second raw material in a larger substance amount (number of moles) than the first raw material in the step of producing the first precursor compound. After that, the first precursor compound and a compound having a structure from which —COOR (R is an alkyl group) is to be derived are reacted with each other. The compound can be produced by this method. As the compound having a structure from which —COOR (R is an alkyl group) is to be derived, it is possible to use, for example, a compound having an ester and a phenolic hydroxyl group such as methyl 4-hydroxybenzoate, ethyl 4-hydroxybenzoate or methyl 6-hydroxy-2-naphthoate.

In a case where the end group having a reactive group in the compound of the present embodiment is —COOH, the compound can be produced by, for example, a method in which a compound in which the end group having a reactive group is —COOR (R is an alkyl group) is produced by the same method as described above and the end group is hydrolyzed.

In a case where the end group having a reactive group in the compound of the present embodiment is —COCl, the compound can be produced by, for example, a method in which a compound in which the end group having a reactive group is —COOH is produced by the same method as described above and the end group and thionyl chloride or oxalyl chloride are reacted with each other.

In a case where the end group having a reactive group in the compound of the present embodiment is —NH₂, the first precursor compound having a monohalogenated methyl group is produced by, for example, using the second raw material in a larger substance amount (number of moles) than the first raw material in the step of producing the first precursor compound. After that, the first precursor compound and a compound having a structure from which —NH₂ is to be derived are reacted with each other. The compound can be produced by this method. As the compound having a structure from which —NH₂ is to be derived, it is possible to use, for example, a compound having an amino group and a phenolic hydroxyl group such as 4-amino-m-cresol or 4-aminophenol.

In a case where the end group having a reactive group in the compound of the present embodiment is —CH═CH₂, the first precursor compound having a monohalogenated methyl group is produced by, for example, using the second raw material in a larger substance amount (number of moles) than the first raw material in the step of producing the first precursor compound. After that, the first precursor compound and a compound having a structure from which —CH═CH₂ is to be derived are reacted with each other. The compound can be produced by this method. As the compound having a structure from which —CH═CH₂ is to be derived, it is possible to use, for example, a compound having a vinyl group directly bonded to an aromatic ring and a phenolic hydroxyl group such as 4-ethenylphenol or 4-ethenyl-2,3,5,7-tetrafluorophenol.

In a case where the end group having a reactive group in the compound of the present embodiment is —CH₂OH, the first precursor compound having a monohalogenated methyl group is produced by, for example, using the second raw material in a larger substance amount (number of moles) than the first raw material in the step of producing the first precursor compound. After that, a bimolecular nucleophilic substitution reaction (S_{N}2 reaction) between the first precursor compound and a hydroxide ion is caused. The compound can be produced by this method.

In a case where the end group having a reactive group in the compound of the present embodiment is —O—COR (R is an alkyl group), the first precursor compound having an —OH group is produced by, for example, using the first raw material in a larger substance amount (number of moles) than the second raw material in the step of producing the first precursor compound. After that, the first precursor compound and a carboxylic acid anhydride such as acetic anhydride or a carboxylic acid chloride such as acetyl chloride are reacted with each other. The compound can be produced by this method.

In a case where the end group having a reactive group in the compound of the present embodiment is the end group represented by Formula (10), the first precursor compound having a monohalogenated methyl group is produced by, for example, using the second raw material in a larger substance amount (number of moles) more than the first raw material in the step of producing the first precursor compound. After that, the first precursor compound and a hydroxide aqueous solution of an alkali metal are reacted with each other to convert a monohalogenated methyl group into a benzyl alcohol group, and the benzyl alcohol group and methacrylic acid chloride are reacted with each other. The compound can be produced by this method.

In a case where the end group having a reactive group in the compound of the present embodiment is the end group represented by Formula (11), the first precursor compound having a monohalogenated methyl group is produced by, for example, using the second raw material in a larger substance amount (number of moles) than the first raw material in the step of producing the first precursor compound. After that, the first precursor compound and a hydroxide aqueous solution of an alkali metal are reacted with each other to convert a monohalogenated methyl group into a benzyl alcohol group, and the benzyl alcohol group and acrylic acid chloride are reacted with each other. The compound can be produced by this method.

In a case where the end group having a reactive group in the compound of the present embodiment is the end group represented by Formula (12), the compound can be produced by, for example, a method in which a compound in which the end group having a reactive group is —OH is produced by the same method as described above and the end group and epichlorohydrin are reacted with each other.

The compound that is obtained by the method for producing the compound of the present embodiment has, between the end groups each having a reactive group that are disposed at both ends respectively, the first structure in which an aromatic cyclic group, an ether oxygen, a methylene group, an aromatic cyclic group, a methylene group, an ether oxygen and an aromatic cyclic group are bonded together in this order and/or the second structure in which an aromatic cyclic group, a methylene group, an ether oxygen, an aromatic cyclic group, an ether oxygen, a methylene group and an aromatic cyclic group are bonded together in this order.

The compound that is obtained by the method for producing the compound of the present embodiment preferably includes a skeleton having a repeating unit composed of one first aromatic cyclic unit and one second aromatic cyclic unit. Furthermore, in the method for producing the compound of the present embodiment, it is preferable to generate a mixture including a plurality of kinds of compounds having different numbers of repeating units at the same time. In the case of producing a polymer using the compound of the present embodiment, there are cases where a plurality of kinds of the compounds of the present embodiment is preferably mixed together and used depending on applications or the like. In the case of generating the mixture including a plurality of kinds of compounds having different numbers of repeating units at the same time, there are cases where a polymer can be efficiently produced without performing a step of mixing the plurality of kinds of compounds of the present embodiment at the time of producing the polymer using the compound of the present embodiment.

In the method for producing the compound of the present embodiment, after the mixture including the plurality of kinds of compounds having different numbers of repeating units is generated at the same time, a single compound having a specific molecular weight may be separated from the mixture of the plurality of kinds of compounds using a well-known method as necessary.

The compound of the present embodiment preferably includes a skeleton having a symmetric structure in which the first aromatic cyclic units and the second aromatic cyclic units are alternately disposed. This skeleton has a structure in which aromatic cyclic groups that are each a mesogenic group developing liquid crystallinity and impart rigidity (the first aromatic cyclic group and the second aromatic cyclic group), and methylene groups and ether oxygens that impart mobility are disposed in a specific order. Due to this fact, the compound of the present embodiment is capable of stabilizing a smectic liquid crystal phase with appropriate mobility intrinsic to the mesogenic groups in spite of having no long side chains which are typically observable in liquid crystal molecules. Therefore, the compound of the present embodiment has high orientation, and a polymerization product which has a smectic liquid crystal structure and is highly thermally conductive due to suppression of the scattering of phonons can be obtained by polymerizing the compound of the present embodiment.

In addition, the compound of the present embodiment has a structure in which end groups each having a reactive group are bonded to both ends of the skeleton. Therefore, a highly thermally conductive resin suitable for an application can be realized by appropriately selecting the kind of the reactive group in the end group depending on the application or the like.

### “Resin Composition”

A resin composition of the present embodiment contains the above-described compound of the present embodiment as a resin component, and the number of the kinds of compound of the present embodiment that the resin composition contains may be only one or two or more.

The resin composition of the present embodiment preferably contains, together with the compound of the present embodiment, a different component as necessary.

The different component can be appropriately determined depending on the application of the resin composition and the kind of the compound of the present embodiment. Examples of the different component include a curing agent, a polymerization accelerator (catalyst), a polymerization initiator, a plasticizer, a resin, a solvent and the like.

In the case of containing, for example, a compound in which the end group having a reactive group is —O—COCH₃ as the compound of the present embodiment, the resin composition of the present embodiment preferably contains terephthalic acid and 4-acetoxybenzoic acid, which is used as necessary, as the different component.

In the case of containing, for example, a compound in which the end group having a reactive group is —COCl as the compound of the present embodiment, the resin composition of the present embodiment preferably contains p-phenylenediamine as the different component.

In the case of containing, for example, a compound in which the end group having a reactive group is —NH₂ as the compound of the present embodiment, the resin composition of the present embodiment preferably contains terephthaloyl dichloride or pyromellitic dianhydride as the different component.

In the case of containing, for example, a compound in which the end group having a reactive group is any of the end group represented by Formula (10) or Formula (11) and —CH—CH₂ as the compound of the present embodiment, the resin composition of the present embodiment can be made to contain the resin composition of the present embodiment, a polymerization initiator and a different monomer having the same kind of reactive group, which is used as necessary.

In the case of containing, for example, a compound in which the end group having a reactive group is any of the end group represented by Formula (10) or Formula (11) and —CH—CH₂ as the compound of the present embodiment, the resin composition of the present embodiment preferably contains a thermally active radical polymerization initiator as the polymerization initiator.

In the case of containing, for example, a compound in which the end group having a reactive group is —CH₂OH as the compound of the present embodiment, the resin composition of the present embodiment preferably contains hexamethylene diisocyanate and/or 4,4′-diisocyanato-3,3′-dimethylphenyl as the different component.

In the case of containing, for example, a compound in which the end group having a reactive group is —OH and/or carboxylic acid ester (—COOR (R is an alkyl group)) as the compound of the present embodiment, a resin composition that can be polymerized by causing an ester exchange reaction using a well-known method may be produced by making the resin composition of the present embodiment contain the resin composition of the present embodiment and a different compound having —OH and/or —COOR (R is an alkyl group).

In the case of containing, for example, a compound in which the end group having a reactive group is the end group represented by Formula (12) as the compound of the present embodiment, the resin composition of the present embodiment can be made to contain the resin composition of the present embodiment, a commercially available epoxy resin, a curing agent and/or a polymerization accelerator.

Examples of the curing agent include a cationic polymerization catalyst such as cyclohexyl p-toluenesulfonate, p-phenylenediamine, 1,5-diaminonaphthalene, hydroquinone, 2,6-dihydroxynaphthalene, phloroglucinol, 4-hydroxybenzoic acid, 6-hydroxy-2-naphthoic acid, 4-aminobenzoic acid, a phenolic resin, polyamideamine, and the like. The amount of the curing agent can be optionally selected.

As the polymerization accelerator, it is possible to use, for example, a basic organic compound having a high boiling point or the like. Specific examples thereof include polymerization accelerators having a boiling point of 200° C. or higher selected from tertiary amines, tertiary phosphines, 4-dimethylaminopyridine (DMAP) or imidazoles and the like. Among these, particularly, 2-ethyl-4-methylimidazole (2E4MZ) and 1-(2-cyanoethyl)-2-phenylimidazole, which are imidazole-based epoxy resin curing agents, are preferably used as the polymerization accelerator due to easiness in handling. The amount of the content of the curing accelerator can be optionally selected.

The resin composition of the present embodiment may contain inorganic particles as necessary. Examples of the inorganic particles include boron nitride particles, magnesium oxide particles, alumina particles, aluminum hydroxide particles, aluminum nitride particles, silica particles and the like. As the inorganic particles, among these, one kind of inorganic particles may be used singly or two or more kinds of inorganic particles may be used in combination.

The amount of the inorganic particles can be optionally selected, but is preferably 200 to 700 parts by mass and more preferably 300 to 600 parts by mass with respect to a total of 100 parts by mass of the resin composition components other than the inorganic particles. The amount of the inorganic particles may be 200 to 500 parts by mass, 200 to 400 parts by mass, 200 to 300 parts by mass, 400 to 500 parts by mass or the like. When the amount of the inorganic particles is 200 parts by mass or more, an effect of improving the thermal conductive property of the resin composition in polymers becomes significant. In addition, when the amount of the inorganic particles is 700 parts by mass or less, sufficient formability can be obtained at the time of forming the resin composition.

The resin composition of the present embodiment may contain a solvent as necessary. Examples of the solvent include ketones such as acetone and methyl ethyl ketone (MEK), alcohols such as methanol, ethanol and isopropanol, aromatic compounds such as toluene and xylene, ethers such as tetrahydrofuran (THF) and 1,3-dioxolane, esters such as ethyl acetate and γ-butyrolactone, amides such as N,N-dimethylformamide (DMF) and N-methylpyrrolidone, and the like. As the solvent, among these, one solvent may be used singly or two or more solvents may be used in combination. The amount of the solvent in the resin composition can be optionally selected as necessary.

A method for producing the resin composition of the present embodiment can be appropriately determined depending on the kind of the end group having a reactive group in the compound of the present embodiment.

For example, the resin composition can be produced by a method in which the compound of the present embodiment and a different component, which is contained as necessary, are mixed together.

Since the resin composition of the present embodiment contains the above-described compound of the present embodiment, a polymer having a high thermal conductivity can be obtained by polymerizing the resin composition.

### “Polymerization Product”

A polymerization product of the present embodiment contains a polymer of the resin composition of the present embodiment.

The shape of the polymerization product of the present embodiment is not particularly limited, and the polymerization product can be formed in a shape of, for example, a sheet shape or a plate shape.

A method for producing the polymerization product of the present embodiment can be appropriately determined depending on the kind of the compound that is contained in the resin composition of the present embodiment.

Specifically, the polymerization product can be produced by, for example, a method in which the compound of the present embodiment is polymerized using a well-known method suited to the end group having a reactive group in the compound of the present embodiment that is contained in the resin composition of the present embodiment.

The polymerization product of the present embodiment contains a polymer obtained by polymerizing the resin composition of the present embodiment and thus has a high thermal conductivity.

### EXAMPLES

### Synthesis of Compounds

### Synthesis Example 1 to Synthesis Example 8

A first raw material shown in Table 1 and a second raw material shown in Table 1 were weighed, respectively, to fractions shown in Table 1 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, twice as many substance amount (number of moles) of potassium carbonate as that of the second raw material was added to the first mixed solution, the refluxed state was held for 12 hours, and a reaction was caused.

After the end of the reaction, the obtained suspension was poured into water and stirred for 30 minutes, and the generated precipitate was filtered and recovered. The recovered precipitate was dried in a vacuum for 12 hours or longer, dissolved in THF (1 L), cooled to 0° C. by adding triethylamine (100 g), and stirred, and acetyl chloride (75 g) was added dropwise thereto while holding the temperature. After that, the solution was gradually heated up to 50° C., stirred for eight hours while holding the temperature, and reacted.

After the end of the reaction, the obtained reaction solution was left to stand in the air such that the temperature reached room temperature, poured into a mixed solvent (1 L) in which water and methanol were mixed in a volume ratio of 1:1 and stirred for 30 minutes, and the generated precipitate was filtered and recovered. The recovered precipitate was dried in a vacuum for 12 hours, thereby obtaining a compound (polyester monomer) of each of Synthesis Example 1 to Synthesis Example 8 in which an end group having a reactive group was —OCOCH₃.

### Synthesis Example 9

A first raw material shown in Table 1 and a second raw material shown in Table 1 were weighed to fractions shown in Table 1 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, twice as many substance amount (number of moles) of potassium carbonate as that of the first raw material was added to the first mixed solution, the refluxed state was held for 12 hours, a reaction was caused, furthermore, methyl 4-hydroxybenzoate (45.6 g, 0.300 mol) and potassium carbonate (82.8 g, 0.600 mol) were added thereto, the refluxed state was held for 12 hours, a reaction was caused, and reflux was performed by adding water for six hours.

After the end of the reaction, the obtained suspension was poured into water, neutralized with hydrochloric acid such that the pH reached in a range of seven to eight and stirred for 30 minutes, and the generated precipitate was filtered and recovered. The recovered precipitate was dried in a vacuum for 12 hours or longer, thereby obtaining a compound of Synthesis Example 9 in which an end group having a reactive group was —COOH.

### Synthesis Example 10

The compound of Synthesis Example 9 was dissolved in N,N-dimethylformamide (DMF) (1 L) and reacted at 90° C. by adding thionyl chloride (70 g) thereto dropwise, and then DMF and thionyl chloride were distilled away at reduced pressure.

The generated solid was heated and dried at 60° C. for 24 hours in a vacuum, thereby obtaining a compound of Synthesis Example 10 in which an end group having a reactive group was —COCl.

Since the compound of Synthesis Example 10 was highly reactive, the measurement (measurement of fractions (mol %) of individual components having different numbers n of repeating units) to be described below was not performed and regarded as the same as in the compound of Synthesis Example 9.

### Synthesis Example 11

A first raw material shown in Table 1 and a second raw material shown in Table 1 were weighed to fractions shown in Table 1 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, twice as many substance amount (number of moles) of potassium carbonate as that of the first raw material was added to the first mixed solution, the refluxed state was held for 12 hours, and a reaction was caused.

4-Amino-m-cresol (36.9 g, 0.300 mol) and potassium carbonate (41.4 g, 0.300 mol) were added to the reaction solution after the end of the reaction, the refluxed state was held for 12 hours, and a reaction was caused.

After the end of the reaction, the obtained suspension was poured into water and stirred for 30 minutes, and the generated precipitate was filtered and recovered. The recovered precipitate was dried in a vacuum for 12 hours or longer, thereby obtaining a compound of Synthesis Example 11 in which an end group having a reactive group was —NH₂.

### Synthesis Example 12 to Synthesis Example 67

A first raw material shown in Table 1 and Table 2 and a second raw material shown in Table 1 and Table 2 were weighed, respectively, to fractions shown in Table 1 and Table 2 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, a 50% aqueous solution of sodium hydroxide which contains 2.1 times as many substance amount (number of moles) of sodium hydroxide as that of the second raw material was added to the first mixed solution, the refluxed state was held for 12 hours, a reaction was caused, and the reaction solution was left to stand in the air such that the temperature reached room temperature.

After the pH of the reaction solution was adjusted to four to six by adding hydrochloric acid to the reaction solution obtained after the end of the reaction, the reaction solution was poured into water and stirred for 30 minutes, and the generated precipitate was filtered and recovered. The recovered precipitate was dried in a vacuum for 12 hours or longer, dissolved in THF (1 L) in a nitrogen atmosphere, cooled to 0° C. by adding triethylamine (100 g), and methacrylic acid chloride (65 g) was added dropwise thereto. After that, the solution was stirred for eight hours while holding the temperature, and reacted.

After the end of the reaction, the temperature of the obtained reaction solution was increased up to room temperature, poured into a mixed solvent (1 L) in which water and methanol were mixed in a volume ratio of 1:1 and stirred for 30 minutes, and the generated precipitate was filtered and recovered. The recovered precipitate was dried in a vacuum for 12 hours, thereby obtaining a compound (methacrylic acid ester monomer) of each of Synthesis Example 12 to Synthesis Example 67 in which an end group having a reactive group was the end group indicated by Formula (10) below.

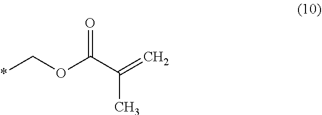 [SMILES: *COC(=O)C(=C)C]

### Synthesis Example 68

A compound (acrylic acid ester monomer) of Synthesis Example 68 in which an end group having a reactive group was the end group indicated by Formula (11) below was obtained using conditions shown in Table 2 in the same manner as in the method for synthesizing the compounds of Synthesis Example 12 to Synthesis Example 67 except that acrylic acid chloride (60 g) was used in place of methacrylic acid chloride.

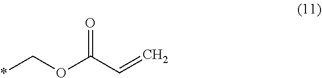 [SMILES: *COC(=O)C=C]

### Synthesis Example 69

A first raw material shown in Table 2 and a second raw material shown in Table 2 were weighed, respectively, to fractions shown in Table 2 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, a 50% aqueous solution of sodium hydroxide which contains twice as many substance amount (number of moles) of sodium hydroxide as that of the second raw material was added to the first mixed solution, the refluxed state was held for 12 hours, a reaction was caused, and the reaction solution was left to stand in the air such that the temperature reached room temperature.

After the pH of the reaction solution was adjusted to four to six by adding hydrochloric acid to the reaction solution obtained after the end of the reaction, the reaction solution was poured into water and stirred for 30 minutes, and the generated precipitate was filtered and recovered. The recovered precipitate was dried in a vacuum for 12 hours or longer and dissolved in THF (1 L), and epichlorohydrin (300 g) was added thereto, thereby producing a second mixed solution. After that, the second mixed solution was refluxed in a nitrogen stream to remove dissolved oxygen in the second mixed solution. Next, a 50% aqueous solution of sodium hydroxide (50 g) was added to the second mixed solution, the refluxed state was held for 12 hours, and a reaction was caused.

After the end of the reaction, the obtained suspension was poured into water and stirred for 30 minutes, and the generated precipitate was filtered and recovered. The recovered precipitate was dried in a vacuum for 12 hours or longer, thereby obtaining a compound (epoxy resin) of Synthesis Example 69 in which an end group in the compound was the end group indicated by Formula (12) below.

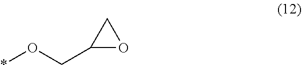 [SMILES: *OCC1CO1]

### Synthesis Example 70

A first raw material shown in Table 2 and a second raw material shown in Table 2 were weighed to fractions shown in Table 2 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, twice as many substance amount (number of moles) of potassium carbonate as that of the first raw material was added to the first mixed solution, the refluxed state was held for 12 hours, and a reaction was caused.

4-Ethenylphenol (36 g, 0.3 mol) and potassium carbonate (41.4 g, 0.300 mol) were added to the reaction solution after the end of the reaction, the refluxed state was held for 12 hours, and a reaction was caused. A suspension obtained after the end of the reaction was poured into a mixed solvent (1 L) in which water and methanol were mixed in a volume ratio of 1:1 and stirred for 30 minutes, and the generated precipitate was filtered and recovered. The recovered precipitate was dried in a vacuum for 12 hours or longer, thereby obtaining a compound of Synthesis Example 70 in which an end group having a reactive group was —CH═CH₂.

### Synthesis Example 71

A first raw material shown in Table 2 and a second raw material shown in Table 2 were weighed to fractions shown in Table 2 in a three-neck flask and dissolved in 1 L of tetrahydrofuran (THL), thereby obtaining a first mixed solution. After that, the first mixed solution was refluxed in a nitrogen stream to remove dissolved oxygen in the first mixed solution. Next, a 50% aqueous solution of sodium hydroxide which contains 2.1 times as many substance amount (number of moles) of sodium hydroxide as that of the second raw material was added to the first mixed solution, the refluxed state was held for 12 hours, a reaction was caused, and the reaction solution was left to stand in the air such that the temperature reached room temperature.

After the pH of the reaction solution was adjusted to four to six by adding hydrochloric acid to the reaction solution obtained after the end of the reaction, the reaction solution was poured into water and stirred for 30 minutes, and the generated precipitate was filtered and recovered. The recovered precipitate was dried in a vacuum for 12 hours or longer, thereby obtaining a compound of Synthesis Example 71 in which an end group having a reactive group was —CH₂OH.

TABLE 1

| | First | Second | First | Second | End group |
|---|---|---|---|---|---|
| | raw | raw | raw ma- | raw ma- | having |
| | ma- | ma- | terial | terial | reactive |
| Compound | terial | terial | (mole) | (mole) | group |
| Synthesis Example 1 | 1-1 | 2-1 | 0.477 | 0.273 | —OCOCH₃ |
| Synthesis Example 2 | 1-1 | 2-1 | 0.450 | 0.300 | —OCOCH₃ |
| Synthesis Example 3 | 1-1 | 2-1 | 0.445 | 0.305 | —OCOCH₃ |
| Synthesis Example 4 | 1-1 | 2-1 | 0.438 | 0.313 | —OCOCH₃ |
| Synthesis Example 5 | 1-1 | 2-1 | 0.424 | 0.326 | —OCOCH₃ |
| Synthesis Example 6 | 1-1 | 2-1 | 0.409 | 0.341 | —OCOCH₃ |
| Synthesis Example 7 | 1-1 | 2-1 | 0.393 | 0.357 | —OCOCH₃ |
| Synthesis Example 8 | 1-1 | 2-1 | 0.643 | 0.107 | —OCOCH₃ |
| Synthesis Example 9 | 1-1 | 2-1 | 0.300 | 0.450 | —COOH |
| Synthesis Example 10 | 1-1 | 2-1 | 0.300 | 0.450 | —COCl |
| Synthesis Example 11 | 1-1 | 2-1 | 0.300 | 0.450 | —NH₂ |
| Synthesis Example 12 | 1-1 | 2-1 | 0.273 | 0.477 | Formula (10) |
| Synthesis Example 13 | 1-1 | 2-1 | 0.300 | 0.450 | Formula (10) |
| Synthesis Example 14 | 1-1 | 2-1 | 0.305 | 0.445 | Formula (10) |
| Synthesis Example 15 | 1-1 | 2-1 | 0.313 | 0.438 | Formula (10) |
| Synthesis Example 16 | 1-1 | 2-1 | 0.326 | 0.424 | Formula (10) |
| Synthesis Example 17 | 1-1 | 2-1 | 0.341 | 0.409 | Formula (10) |
| Synthesis Example 18 | 1-1 | 2-1 | 0.357 | 0.393 | Formula (10) |
| Synthesis Example 19 | 1-1 | 2-1 | 0.107 | 0.643 | Formula (10) |
| Synthesis Example 20 | 1-2 | 2-1 | 0.300 | 0.450 | Formula (10) |
| Synthesis Example 21 | 1-2 | 2-2 | 0.300 | 0.450 | Formula (10) |
| Synthesis Example 22 | 1-1 | 2-2 | 0.300 | 0.450 | Formula (10) |
| Synthesis Example 23 | 1-3 | 2-1 | 0.300 | 0.450 | Formula (10) |
| Synthesis Example 24 | 1-2 | 2-3 | 0.300 | 0.450 | Formula (10) |
| Synthesis Example 25 | 1-3 | 2-3 | 0.300 | 0.450 | Formula (10) |
| Synthesis Example 26 | 1-4 | 2-1 | 0.300 | 0.450 | Formula (10) |
| Synthesis Example 27 | 1-5 | 2-1 | 0.300 | 0.450 | Formula (10) |
| Synthesis Example 28 | 1-6 | 2-1 | 0.300 | 0.450 | Formula (10) |
| Synthesis Example 29 | 1-7 | 2-1 | 0.300 | 0.450 | Formula (10) |
| Synthesis Example 30 | 1-8 | 2-1 | 0.300 | 0.450 | Formula (10) |
| Synthesis Example 31 | 1-9 | 2-1 | 0.300 | 0.450 | Formula (10) |
| Synthesis Example 32 | 1-5 | 2-2 | 0.300 | 0.450 | Formula (10) |
| Synthesis Example 33 | 1-6 | 2-2 | 0.300 | 0.450 | Formula (10) |
| Synthesis Example 34 | 1-7 | 2-2 | 0.300 | 0.450 | Formula (10) |
| Synthesis Example 35 | 1-8 | 2-2 | 0.300 | 0.450 | Formula (10) |

TABLE 2

| | First | Second | First | Second | End group |
|---|---|---|---|---|---|
| | raw | raw | raw ma- | raw ma- | having |
| | ma- | ma- | terial | terial | reactive |
| Compound | terial | terial | (mole) | (mole) | group |
| Synthesis Example 36 | 1-9 | 2-2 | 0.300 | 0.450 | Formula (10) |
| Synthesis Example 37 | 1-1 | 2-4 | 0.300 | 0.450 | Formula (10) |
| Synthesis Example 38 | 1-1 | 2-5 | 0.300 | 0.450 | Formula (10) |
| Synthesis Example 39 | 1-1 | 2-6 | 0.300 | 0.450 | Formula (10) |
| Synthesis Example 40 | 1-1 | 2-7 | 0.300 | 0.450 | Formula (10) |
| Synthesis Example 41 | 1-6 | 2-4 | 0.300 | 0.450 | Formula (10) |
| Synthesis Example 42 | 1-6 | 2-5 | 0.300 | 0.450 | Formula (10) |
| Synthesis Example 43 | 1-10 | 2-1 | 0.300 | 0.450 | Formula (10) |
| Synthesis Example 44 | 1-11 | 2-1 | 0.300 | 0.450 | Formula (10) |
| Synthesis Example 45 | 1-12 | 2-1 | 0.300 | 0.450 | Formula (10) |
| Synthesis Example 46 | 1-2 | 2-8 | 0.300 | 0.450 | Formula (10) |
| Synthesis Example 47 | 1-2 | 2-9 | 0.300 | 0.450 | Formula (10) |
| Synthesis Example 48 | 1-2 | 2-10 | 0.300 | 0.450 | Formula (10) |
| Synthesis Example 49 | 1-10 | 2-8 | 0.300 | 0.450 | Formula (10) |
| Synthesis Example 50 | 1-11 | 2-9 | 0.300 | 0.450 | Formula (10) |
| Synthesis Example 51 | 1-12 | 2-10 | 0.300 | 0.450 | Formula (10) |
| Synthesis Example 52 | 1-1 | 2-11 | 0.300 | 0.450 | Formula (10) |
| Synthesis Example 53 | 1-1 | 2-12 | 0.300 | 0.450 | Formula (10) |
| Synthesis Example 54 | 1-1 | 2-13 | 0.300 | 0.450 | Formula (10) |
| Synthesis Example 55 | 1-2 | 2-14 | 0.300 | 0.450 | Formula (10) |
| Synthesis Example 56 | 1-13 | 2-1 | 0.300 | 0.450 | Formula (10) |
| Synthesis Example 57 | 1-13 | 2-14 | 0.300 | 0.450 | Formula (10) |
| Synthesis Example 58 | 1-13 | 2-13 | 0.300 | 0.450 | Formula (10) |
| Synthesis Example 59 | 1-14 | 2-14 | 0.300 | 0.450 | Formula (10) |
| Synthesis Example 60 | 1-14 | 2-13 | 0.300 | 0.450 | Formula (10) |
| Synthesis Example 61 | 1-15 | 2-1 | 0.300 | 0.450 | Formula (10) |
| Synthesis Example 62 | 1-15 | 2-14 | 0.300 | 0.450 | Formula (10) |
| Synthesis Example 63 | 1-15 | 2-15 | 0.300 | 0.450 | Formula (10) |
| Synthesis Example 64 | 1-16 | 2-1 | 0.300 | 0.450 | Formula (10) |
| Synthesis Example 65 | 1-16 | 2-14 | 0.300 | 0.450 | Eormula (10) |
| Synthesis Example 66 | 1-17 | 2-1 | 0.300 | 0.450 | Formula (10) |
| Synthesis Example 67 | 1-17 | 2-13 | 0.300 | 0.450 | Formula (10) |
| Synthesis Example 68 | 1-1 | 2-1 | 0.300 | 0.450 | Formula (11) |
| Synthesis Example 69 | 1-1 | 2-1 | 0.450 | 0.300 | Formula (12) |
| Synthesis Example 70 | 1-1 | 2-1 | 0.300 | 0.450 | —CH═CH₂ |
| Synthesis Example 71 | 1-1 | 2-1 | 0.300 | 0.450 | —CH₂OH |

1-1 to 1-17 in the columns “first raw material” in Table 1 and Table 2 are the following compounds.

### “First Raw Materials”

(1-1) Methylhydroquinone
(1-2) Hydroquinone
(1-3) Tetramethylhydroquinone
(1-4) Trimethylhydroquinone
(1-5) 2-(Trifluoromethyl)-1,4-benzenediol
(1-6) Fluorohydroquinone
(1-7) Chlorohydroquinone
(1-8) Bromohydroquinone
(1-9) 2,5-Dihydroxynitrobenzene
(1-10) Tetrafluorohydraquinone
(1-11) Tetrachlorohydroquinone
(1-12) Tetrabromohydroquinone
(1-13) 2,6-Dihydroxynaphthalene
(1-14) 1,5-Dihydroxynaphthalene
(1-15) 4,4′-Dihydroxybiphenyl
(1-16) 3,3′,5,5′-Tetramethylbiphenyl-4,4′-diol
(1-17) 1,4-Dihydroxynaphthalene

2-1 to 2-15 in the columns “second raw material” in Table 1 and Table 2 are the following compounds.

### “Second Raw Materials”

(2-1) α,α′-p-Dichloroxylene
(2-2) 1,4-Bis(chloromethyl)-2-methylbenzene
(2-3) 3,6-Bis(chloromethyl)durene
(2-4) 1,4-Bis(bromomethyl)-2-fluorobenzene
(2-5) 1,4-Bis(bromomethyl)-2-chlorobenzene
(2-6) 2-Bromo-1,4-bis(bromomethyl)benzene
(2-7) 1,4-Bis(chloromethyl)-2-nitrobenzene
(2-8) 1,4-Bis(bromomethyl)-2,3,5,6-tetrafluorobenzene
(2-9) α,α′,2,3,5,6-Hexachloro-p-xylene
(2-10) 1,2,4,5-Tetrabromo-3,6-bis-bromomethyl-benzene
(2-11) 1,2-Dibromo-3,6-bis(chloromethyl)-4,5-dimethylbenzene
(2-12) 1,4-Bis(bromomethyl)-2,5-dimethylbenzene
(2-13) 4,4′-Bis(chloromethyl)biphenyl
(2-14) 2,6-Bis(bromomethyl)naphthalene
(2-15) 1,5-Bis(chloromethyl)naphthalene

For the compounds of Synthesis Example 1 to Synthesis Example 71 obtained as described above, the respective structures were confirmed by a method described below using preparative gel permeation chromatography (GPC) and matrix-assisted laser desorption ionization-time of flight mass spectrometry (MALDI-TOF MS).

First, the compounds of Synthesis Example 1 to Synthesis Example 71 were analyzed, respectively, using preparative gel permeation chromatography (GPC) (manufactured by Shimadzu Corporation), a GPC column (GPCKF-2001 (manufactured by SHOWA DENKO K.K.) as a column and THF as an eluent. As a result, it was found that the compounds of Synthesis Example 1 to Synthesis Example 71 were all mixtures composed of a plurality of compounds having different molecular weights.

### (Measurement of Fractions (Mol %) of Individual Components Having Different Numbers n of Repeating Units)

Each of the compounds of Synthesis Example 1 to Synthesis Example 71 was separated into components (compounds) having different molecular weights using the preparative gel permeation chromatography (GPC). In addition, for the individual components having different molecular weights, the masses were measured in a cation detection mode using matrix-assisted laser desorption ionization-time of flight mass spectrometry (MALDI-TOF MS) (manufactured by JEOL Ltd.), and the value of the peak having the strongest intensity was regarded as the molecular weight. In addition, the measurement results (measured values) of the obtained molecular weights and the molecular weights (calculated values) of the presumed molecular structures were cross-checked, thereby identifying the compounds of Synthesis Example 1 to Synthesis Example 71, respectively.

The measurement results (measured values) of the obtained molecular weights and the molecular weights (calculated values) of the presumed molecular structures are shown in Table 3 to Table 8. In addition, the structures of the identified compounds of Synthesis Example 1 to Synthesis Example 71 will be shown below.

TABLE 3

| Compound | Number of repeating units (n) | 0 | 1 | 2 | 3 | 4 | 5 | 6 |
|---|---|---|---|---|---|---|---|---|
| Synthesis | Calculated molecular weight value | 434.45 | 660.76 | 887.08 | 1113.39 | 1339.71 | | |
| Example 1 | Measured molecular weight value | 433 | 659 | 886 | 1112 | 1338 | | |
| Synthesis | Calculated molecular weight value | 434.45 | 660.76 | 887.08 | 1113.39 | 1339.71 | 1566.02 | 1792.34 |
| Example 2 | Measured molecular weight value | 433 | 659 | 886 | 1112 | 1338 | 1565 | 1791 |
| Synthesis | Calculated molecular weight value | | 660.76 | 887.08 | 1113.39 | 1339.71 | 1566.02 | 1792.34 |
| Example 3 | Measured molecular weight value | | 659 | 886 | 1112 | 1338 | 1565 | 1791 |
| Synthesis | Calculated molecular weight value | | | 887.08 | 1113.39 | 1339.71 | 1566.02 | 1792.34 |
| Example 4 | Measured molecular weight value | | | 886 | 1112 | 1338 | 1565 | 1791 |
| Synthesis | Calculated molecular weight value | | | | | | | 1792.34 |
| Example 5 | Measured molecular weight value | | | | | | | 1791 |
| Synthesis | Calculated molecular weight value | | | | | | | |
| Example 6 | Measured molecular weight value | | | | | | | |
| Synthesis | Calculated molecular weight value | | | | | | | |
| Example 7 | Measured molecular weight value | | | | | | | |
| Synthesis | Calculated molecular weight value | 434.45 | 660.76 | | | | | |
| Example 8 | Measured molecular weight value | 433 | 659 | | | | | |
| Synthesis | Calculated molecular weight value | 378.38 | 604.66 | 830.93 | 1057.21 | 1283.48 | 1509.76 | 1736.03 |
| Example 9, 10 | Measured molecular weight value | 377 | 603 | 829 | 1056 | 1282 | 1508 | 1735 |
| Synthesis | Calculated molecular weight value | 348.45 | 574.72 | 801.00 | 1027.27 | 1253.55 | 1479.82 | 1706.10 |
| Example 11 | Measured molecular weight value | 347 | 573 | 799 | 1026 | 1252 | 1478 | 1705 |
| Synthesis | Calculated molecular weight value | 498.62 | 726.87 | 955.11 | 1183.36 | 1411.61 | | |
| Example 12 | Measured molecular weight value | 497 | 725 | 954 | 1182 | 1410 | | |
| Synthesis | Calculated molecular weight value | 498.62 | 726.87 | 955.11 | 1183.36 | 1411.61 | 1639.85 | 1868.10 |
| Example 13 | Measured molecular weight value | 497 | 725 | 954 | 1182 | 1410 | 1638 | 1867 |
| Synthesis | Calculated molecular weight value | | 726.87 | 955.11 | 1183.36 | 1411.61 | 1639.85 | 1868.10 |
| Example 14 | Measured molecular weight value | | 725 | 954 | 1182 | 1410 | 1638 | 1867 |
| Synthesis | Calculated molecular weight value | | | 955.11 | 1183.36 | 1411.61 | 1639.85 | 1868.10 |
| Example 15 | Measured molecular weight value | | | 954 | 1182 | 1410 | 1638 | 1867 |
| Synthesis | Calculated molecular weight value | | | | | | | 1868.10 |
| Example 16 | Measured molecular weight value | | | | | | | 1867 |
| Synthesis | Calculated molecular weight value | | | | | | | |
| Example 17 | Measured molecular weight value | | | | | | | |
| Synthesis | Calculated molecular weight value | | | | | | | |
| Example 18 | Measured molecular weight value | | | | | | | |
| Synthesis | Calculated molecular weight value | 484.59 | 696.84 | | | | | |
| Example 19 | Measured molecular weight value | 483 | 695 | | | | | |

TABLE 4

| Compound | Number of repeating units (n) | 7 | 8 | 9 | 10 | 11 | 12 | 13 |
|---|---|---|---|---|---|---|---|---|
| Synthesis | Calculated molecular weight value | | | | | | | |
| Example 1 | Measured molecular weight value | | | | | | | |
| Synthesis | Calculated molecular weight value | | | | | | | |
| Example 2 | Measured molecular weight value | | | | | | | |
| Synthesis | Calculated molecular weight value | 2018.65 | | | | | | |
| Example 3 | Measured molecular weight value | 2017 | | | | | | |
| Synthesis | Calculated molecular weight value | 2018.65 | 2244.97 | 2471.28 | 2697.60 | | | |
| Example 4 | Measured molecular weight value | 2017 | 2243 | 2470 | 2696 | | | |
| Synthesis | Calculated molecular weight value | 2018.65 | 2244.97 | 2471.28 | 2697.60 | | | |
| Example 5 | Measured molecular weight value | 2017 | 2243 | 2470 | 2696 | | | |
| Synthesis | Calculated molecular weight value | | 2244.97 | 2471.28 | 2697.60 | 2923.91 | 3150.23 | 3376.54 |
| Example 6 | Measured molecular weight value | | 2243 | 2470 | 2696 | 2922 | 3149 | 3375 |
| Synthesis | Calculated molecular weight value | | | | | | 3150.23 | 3376.54 |
| Example 7 | Measured molecular weight value | | | | | | 3149 | 3375 |
| Synthesis | Calculated molecular weight value | | | | | | | |
| Example 8 | Measured molecular weight value | | | | | | | |
| Synthesis | Calculated molecular weight value | | | | | | | |
| Example 9, 10 | Measured molecular weight value | | | | | | | |
| Synthesis | Calculated molecular weight value | | | | | | | |
| Example 11 | Measured molecular weight value | | | | | | | |
| Synthesis | Calculated molecular weight value | | | | | | | |
| Example 12 | Measured molecular weight value | | | | | | | |
| Synthesis | Calculated molecular weight value | | | | | | | |
| Example 13 | Measured molecular weight value | | | | | | | |
| Synthesis | Calculated molecular weight value | 2096.35 | | | | | | |
| Example 14 | Measured molecular weight value | 2095 | | | | | | |
| Synthesis | Calculated molecular weight value | 2096.35 | 2324.60 | 2552.84 | 2781.09 | | | |
| Example 15 | Measured molecular weight value | 2095 | 2323 | 2551 | 2780 | | | |
| Synthesis | Calculated molecular weight value | 2096.35 | 2324.60 | 2552.84 | 2781.09 | 3009.34 | | |
| Example 16 | Measured molecular weight value | 2095 | 2323 | 2551 | 2780 | 3008 | | |
| Synthesis | Calculated molecular weight value | | 2324.60 | 2552.84 | 2781.09 | 3009.34 | 3237.58 | 3465.83 |
| Example 17 | Measured molecular weight value | | 2323 | 2551 | 2780 | 3008 | 3236 | 3464 |
| Synthesis | Calculated molecular weight value | | | | | | 3237.58 | 3465.83 |
| Example 18 | Measured molecular weight value | | | | | | 3236 | 3464 |
| Synthesis | Calculated molecular weight value | | | | | | | |
| Example 19 | Measured molecular weight value | | | | | | | |

TABLE 5

| Compound | Number of repeating units (n) | 14 | 15 | 16 | 17 | 18 | 19 | 20 |
|---|---|---|---|---|---|---|---|---|
| Synthesis | Calculated molecular weight value | | | | | | | |
| Example 1 | Measured molecular weight value | | | | | | | |
| Synthesis | Calculated molecular weight value | | | | | | | |
| Example 2 | Measured molecular weight value | | | | | | | |
| Synthesis | Calculated molecular weight value | | | | | | | |
| Example 3 | Measured molecular weight value | | | | | | | |
| Synthesis | Calculated molecular weight value | | | | | | | |
| Example 4 | Measured molecular weight value | | | | | | | |
| Synthesis | Calculated molecular weight value | | | | | | | |
| Example 5 | Measured molecular weight value | | | | | | | |
| Synthesis | Calculated molecular weight value | 3602.86 | 3829.17 | 4055.49 | | | | |
| Example 6 | Measured molecular weight value | 3601 | 3828 | 4054 | | | | |
| Synthesis | Calculated molecular weight value | 3602.86 | 3829.17 | 4055.49 | 4281.80 | 4508.12 | 4734.43 | 4960.75 |
| Example 7 | Measured molecular weight value | 3601 | 3828 | 4054 | 4280 | 4507 | 4733 | 4959 |
| Synthesis | Calculated molecular weight value | | | | | | | |
| Example 8 | Measured molecular weight value | | | | | | | |
| Synthesis | Calculated molecular weight value | | | | | | | |
| Example 9, 10 | Measured molecular weight value | | | | | | | |
| Synthesis | Calculated molecular weight value | | | | | | | |
| Example 11 | Measured molecular weight value | | | | | | | |
| Synthesis | Calculated molecular weight value | | | | | | | |
| Example 12 | Measured molecular weight value | | | | | | | |
| Synthesis | Calculated molecular weight value | | | | | | | |
| Example 13 | Measured molecular weight value | | | | | | | |
| Synthesis | Calculated molecular weight value | | | | | | | |
| Example 14 | Measured molecular weight value | | | | | | | |
| Synthesis | Calculated molecular weight value | | | | | | | |
| Example 15 | Measured molecular weight value | | | | | | | |
| Synthesis | Calculated molecular weight value | | | | | | | |
| Example 16 | Measured molecular weight value | | | | | | | |
| Synthesis | Calculated molecular weight value | 3694.08 | 3922.32 | 4150.57 | | | | |
| Example 17 | Measured molecular weight value | 3693 | 3921 | 4149 | | | | |
| Synthesis | Calculated molecular weight value | 3694.08 | 3922.32 | 4150.57 | 4378.82 | 4607.07 | 4835.31 | 5063.56 |
| Example 18 | Measured molecular weight value | 3693 | 3921 | 4149 | 4377 | 4606 | 4834 | 5062 |
| Synthesis | Calculated molecular weight value | | | | | | | |
| Example 19 | Measured molecular weight value | | | | | | | |

TABLE 6

| | | | | | | | | |
|---|---|---|---|---|---|---|---|---|
| Compound | Number of repeating units (n) | 0 | 1 | 2 | 3 | 4 | 5 | 6 |

| | | | | | | | |
|---|---|---|---|---|---|---|---|
| Synthesis | Calculated molecular weight value | 512.65 | 738.92 | 965.20 | 1191.47 | 1417.75 | 1644.02 |
| Example 20 | Measured molecular weight value | 511 | 737 | 964 | 1190 | 1416 | 1643 |
| Synthesis | Calculated molecular weight value | 508.67 | 768.95 | 1029.22 | 1289.50 | 1549.77 | 1810.04 |
| Example 21 | Measured molecular weight value | 507 | 767 | 1028 | 1288 | 1548 | 1809 |
| Synthesis | Calculated molecular weight value | 540.70 | 811.03 | 1081.36 | 1351.68 | 1622.01 | |
| Example 22 | Measured molecular weight value | 539 | 810 | 1080 | 1350 | 1621 | |
| Synthesis | Calculated molecular weight value | 528.65 | 768.95 | 1009.25 | 1249.55 | 1489.85 | 1730.16 |
| Example 23 | Measured molecular weight value | 527 | 767 | 1008 | 1248 | 1488 | 1729 |
| Synthesis | Calculated molecular weight value | 596.81 | 867.14 | 1137.46 | 1407.79 | 1678.12 | |
| Example 24 | Measured molecular weight value | 595 | 866 | 1136 | 1406 | 1677 | |
| Synthesis | Calculated molecular weight value | 652.92 | 979.35 | 1305.79 | 1632.22 | 1958.66 | 2285.10 |
| Example 25 | Measured molecular weight value | 651 | 978 | 1304 | 1631 | 1957 | 2284 |
| Synthesis | Calculated molecular weight value | 528.645 | 782.974 | 1037.303 | 1291.632 | 1545.961 | 1800.29 |
| Example 26 | Measured molecular weight value | 527 | 781 | 1036 | 1290 | 1544 | 1799 |
| Synthesis | Calculated molecular weight value | 526.67 | 782.97 | 1039.28 | 1295.58 | 1551.88 | 1808.18 |
| Example 27 | Measured molecular weight value | 525 | 781 | 1038 | 1294 | 1550 | 1807 |
| Synthesis | Calculated molecular weight value | 552.59 | 834.81 | 1117.03 | 1399.24 | 1681.46 | 1963.68 |
| Example 28 | Measured molecular weight value | 551 | 833 | 1116 | 1398 | 1680 | 1962 |
| Synthesis | Calculated molecular weight value | 504.55 | 734.79 | 965.03 | 1195.27 | 1425.51 | 1655.75 |
| Example 29 | Measured molecular weight value | 503 | 733 | 964 | 1194 | 1424 | 1654 |
| Synthesis | Calculated molecular weight value | 519.03 | 767.70 | 1016.37 | 1265.04 | 1513.71 | 1762.38 |
| Example 30 | Measured molecular weight value | 518 | 766 | 1015 | 1264 | 1512 | 1761 |
| Synthesis | Calculated molecular weight value | 565.41 | 856.60 | 1147.80 | 1438.99 | 1730.19 | 2021.38 |
| Example 31 | Measured molecular weight value | 564 | 855 | 1146 | 1437 | 1729 | 2020 |
| Synthesis | Calculated molecular weight value | 529.59 | 788.81 | 1048.02 | 1307.24 | 1566.46 | 1825.67 |
| Example 32 | Measured molecular weight value | 528 | 787 | 1047 | 1306 | 1565 | 1824 |
| Synthesis | Calculated molecular weight value | 532.60 | 776.87 | 1021.14 | 1265.41 | 1509.68 | 1753.95 |
| Example 33 | Measured molecular weight value | 531 | 775 | 1020 | 1264 | 1508 | 1752 |
| Synthesis | Calculated molecular weight value | 549.06 | 709.78 | 870.50 | 1031.22 | 1191.94 | 1352.66 |
| Example 34 | Measured molecular weight value | 548 | 708 | 869 | 1030 | 1190 | 1351 |
| Synthesis | Calculated molecular weight value | 593.51 | 898.69 | 1203.86 | 1509.03 | 1814.20 | 2119.37 |
| Example 35 | Measured molecular weight value | 592 | 897 | 1202 | 1508 | 1813 | 2118 |

TABLE 7

| | | | | | | | | |
|---|---|---|---|---|---|---|---|---|
| Compound | Number of repeating units (n) | 0 | 1 | 2 | 3 | 4 | 5 | 6 |

| | | | | | | | |
|---|---|---|---|---|---|---|---|
| Synthesis | Calculated molecular weight value | 591.54 | 898.69 | 1205.83 | 1512.97 | 1820.11 | 2127.26 |
| Example 36 | Measured molecular weight value | 590 | 897 | 1204 | 1511 | 1819 | 2126 |
| Synthesis | Calculated molecular weight value | 536.57 | 780.84 | 1025.10 | 1269.37 | 1513.63 | 1757.90 |
| Example 37 | Measured molecular weight value | 535 | 779 | 1024 | 1268 | 1512 | 1756 |
| Synthesis | Calculated molecular weight value | 569.48 | 830.19 | 1090.90 | 1351.61 | 1612.32 | 1873.03 |
| Example 38 | Measured molecular weight value | 568 | 829 | 1089 | 1350 | 1611 | 1872 |
| Synthesis | Calculated molecular weight value | 658.38 | 963.55 | 1268.73 | 1573.90 | 1879.07 | 2184.24 |
| Example 39 | Measured molecular weight value | 657 | 962 | 1267 | 1572 | 1878 | 2183 |
| Synthesis | Calculated molecular weight value | 656.41 | 963.55 | 1270.70 | 1577.84 | 1884.98 | 2192.13 |
| Example 40 | Measured molecular weight value | 655 | 962 | 1269 | 1576 | 1883 | 2191 |
| Synthesis | Calculated molecular weight value | 540.54 | 788.76 | 1036.99 | 1285.22 | 1533.45 | 1781.68 |
| Example 41 | Measured molecular weight value | 539 | 787 | 1035 | 1284 | 1532 | 1780 |
| Synthesis | Calculated molecular weight value | 573.45 | 838.12 | 1102.79 | 1367.46 | 1632.13 | 1896.80 |
| Example 42 | Measured molecular weight value | 572 | 837 | 1101 | 1366 | 1631 | 1895 |
| Synthesis | Calculated molecular weight value | 558.53 | 842.74 | 1126.94 | 1411.15 | 1695.36 | 1979.57 |
| Example 43 | Measured molecular weight value | 557 | 841 | 1125 | 1410 | 1694 | 1978 |
| Synthesis | Calculated molecular weight value | 624.34 | 974.35 | 1324.36 | 1674.37 | 2024.38 | 2374.39 |
| Example 44 | Measured molecular weight value | 623 | 973 | 1323 | 1673 | 2023 | 2373 |
| Synthesis | Calculated molecular weight value | 802.15 | 1329.98 | 1857.81 | 2385.63 | 2913.46 | 3441.28 |
| Example 45 | Measured molecular weight value | 801 | 1328 | 1856 | 2384 | 2912 | 3440 |
| Synthesis | Calculated molecular weight value | 630.49 | 914.70 | 1198.90 | 1483.11 | 1767.31 | 2051.51 |
| Example 46 | Measured molecular weight value | 629 | 913 | 1197 | 1482 | 1766 | 2050 |
| Synthesis | Calculated molecular weight value | 762.11 | 1112.12 | 1462.13 | 1812.14 | 2162.15 | 2512.16 |
| Example 47 | Measured molecular weight value | 761 | 1111 | 1461 | 1811 | 2161 | 2511 |
| Synthesis | Calculated molecular weight value | 1117.74 | 1645.56 | 2173.39 | 2701.22 | 3229.04 | 3756.87 |
| Example 48 | Measured molecular weight value | 1116 | 1644 | 2172 | 2700 | 3228 | 3755 |
| Synthesis | Calculated molecular weight value | 702.46 | 1058.62 | 1414.79 | 1770.95 | 2127.12 | 2483.28 |
| Example 49 | Measured molecular weight value | 701 | 1057 | 1413 | 1769 | 2126 | 2482 |
| Synthesis | Calculated molecular weight value | 899.88 | 1387.65 | 1875.42 | 2363.19 | 2850.96 | 3338.73 |
| Example 50 | Measured molecular weight value | 898 | 1386 | 1874 | 2362 | 2849 | 3337 |
| Synthesis | Calculated molecular weight value | 1433.35 | 2276.73 | 3120.11 | 3963.49 | 4806.87 | 5650.25 |
| Example 51 | Measured molecular weight value | 1432 | 2275 | 3119 | 3962 | 4805 | 5649 |

TABLE 8

| Compound | Number of repeating units (n) | 0 | 1 | 2 | 3 | 4 | 5 | 6 |
|---|---|---|---|---|---|---|---|---|
| Synthesis | Calculated molecular weight value | 872.29 | 1284.40 | 1696.52 | 2108.63 | 2520.75 | 2932.86 | |
| Example 52 | Measured molecular weight value | 871 | 1283 | 1695 | 2107 | 2519 | 2931 | |
| Synthesis | Calculated molecular weight value | 556.70 | 811.03 | 1065.36 | 1319.69 | 1574.02 | 1828.34 | |
| Example 53 | Measured molecular weight value | 555 | 810 | 1064 | 1318 | 1573 | 1827 | |
| Synthesis | Calculated molecular weight value | 652.79 | 955.16 | 1257.53 | 1559.91 | 1862.28 | 2164.65 | |
| Example 54 | Measured molecular weight value | 651 | 954 | 1256 | 1558 | 1861 | 2163 | |
| Synthesis | Calculated molecular weight value | 516.64 | 848.99 | 1181.35 | 1513.70 | 1846.06 | 2178.41 | |
| Example 55 | Measured molecular weight value | 515 | 847 | 1180 | 1512 | 1845 | 2177 | |
| Synthesis | Calculated molecular weight value | 536.62 | 796.96 | 1057.30 | 1317.63 | 1577.97 | 1838.30 | |
| Example 56 | Measured molecular weight value | 535 | 795 | 1056 | 1316 | 1576 | 1837 | |
| Synthesis | Calculated molecular weight value | 636.74 | 949.11 | 1261.48 | 1573.85 | 1886.22 | 2198.58 | |
| Example 57 | Measured molecular weight value | 635 | 948 | 1260 | 1572 | 1885 | 2197 | |
| Synthesis | Calculated molecular weight value | 696.82 | 1039.23 | 1381.63 | 1724.04 | 2066.44 | 2408.85 | |
| Example 58 | Measured molecular weight value | 695 | 1038 | 1380 | 1723 | 2065 | 2407 | |
| Synthesis | Calculated molecular weight value | 636.74 | 949.11 | 1261.48 | 1573.85 | 1886.22 | 2198.58 | |
| Example 59 | Measured molecular weight value | 635 | 948 | 1260 | 1572 | 1885 | 2197 | |
| Synthesis | Calculated molecular weight value | 696.82 | 1039.23 | 1381.63 | 1724.04 | 2066.44 | 2408.85 | |
| Example 60 | Measured molecular weight value | 695 | 1038 | 1380 | 1723 | 2065 | 2407 | |
| Synthesis | Calculated molecular weight value | 566.69 | 857.06 | 1147.42 | 1437.79 | 1728.15 | | |
| Example 61 | Measured molecular weight value | 565 | 856 | 1146 | 1436 | 1727 | | |
| Synthesis | Calculated molecular weight value | 662.78 | 1001.19 | 1339.59 | 1678.00 | 2016.41 | 2354.81 | |
| Example 62 | Measured molecular weight value | 661 | 1000 | 1338 | 1677 | 2015 | 2353 | |
| Synthesis | Calculated molecular weight value | 662.78 | 1001.19 | 1339.59 | 1678.00 | 2016.41 | 2354.81 | |
| Example 63 | Measured molecular weight value | 661 | 1000 | 1338 | 1677 | 2015 | 2353 | |
| Synthesis | Calculated molecular weight value | 622.80 | 969.27 | 1315.74 | 1662.21 | 2008.68 | 2355.15 | |
| Example 64 | Measured molecular weight value | 621 | 968 | 1314 | 1661 | 2007 | 2354 | |
| Synthesis | Calculated molecular weight value | 718.89 | 1113.40 | 1507.92 | 1902.43 | 2296.95 | 2691.46 | |
| Example 65 | Measured molecular weight value | 717 | 1112 | 1506 | 1901 | 2295 | 2690 | |
| Synthesis | Calculated molecular weight value | 536.62 | 796.96 | 1057.30 | 1317.63 | 1577.97 | 1838.30 | |
| Example 66 | Measured molecular weight value | 535 | 795 | 1056 | 1316 | 1576 | 1837 | |
| Synthesis | Calculated molecular weight value | 696.82 | 1039.23 | 1381.63 | 1724.04 | 2066.44 | 2408.85 | |
| Example 67 | Measured molecular weight value | 695 | 1038 | 1380 | 1723 | 2065 | 2407 | |
| Synthesis | Calculated molecular weight value | 476.47 | 704.66 | 932.85 | 1161.04 | 1389.23 | 1617.42 | 1845.62 |
| Example 68 | Measured molecular weight value | 475 | 703 | 931 | 1160 | 1388 | 1616 | 1844 |
| Synthesis | Calculated molecular weight value | 464.36 | 692.55 | 920.74 | 1148.93 | 1377.12 | 1605.31 | 1833.50 |
| Example 69 | Measured molecular weight value | 463 | 691 | 919 | 1147 | 1376 | 1604 | 1832 |
| Synthesis | Calculated molecular weight value | 362.413 | 590.604 | 818.795 | 1046.986 | 1275.177 | 1503.368 | 1731.559 |
| Example 70 | Measured molecular weight value | 361 | 589 | 817 | 1045 | 1274 | 1502 | 1730 |
| Synthesis | Calculated molecular weight value | 368.375 | 596.566 | 824.757 | 1052.948 | 1281.139 | 1509.33 | 1737.521 |
| Example 71 | Measured molecular weight value | 367 | 595 | 823 | 1051 | 1280 | 1508 | 1736 |

The compounds of Synthesis Example 1 to Synthesis Example 8 are indicated by General Formula (A) shown above (in Formula (A), n is a numerical value shown in Table 3 to Table 5.).

The compound of Synthesis Example 9 is indicated by General Formula (E) below (in Formula (E), n is a numerical value shown in Table 3 to Table 5. * is —H.). The compound of Synthesis Example 10 is indicated by General Formula (E) below (in Formula (E), n is a numerical value shown in Table 3 to Table 5. * is —Cl.).

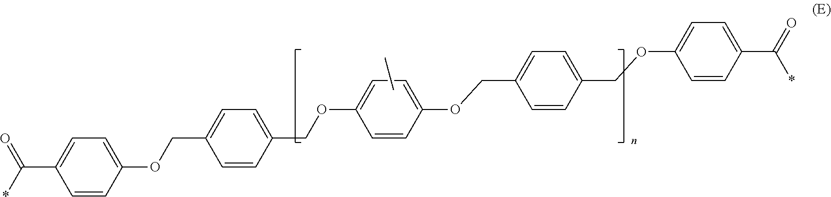

The compound of Synthesis Example 11 is indicated by General Formula (F).

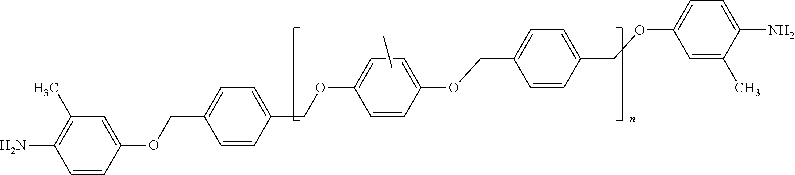

(In Formula (F), n is a numerical value shown in Table 3 to Table 5.)

The compounds of Synthesis Example 12 to Synthesis Example 19 are indicated by General Formula (B) shown above (in Formula (B), n is a numerical value shown in Table 3 to Table 5.).

The compounds of Synthesis Example 20 to Synthesis Example 22 and Synthesis Example 27 to Synthesis Example 42 are indicated by General Formula (H).

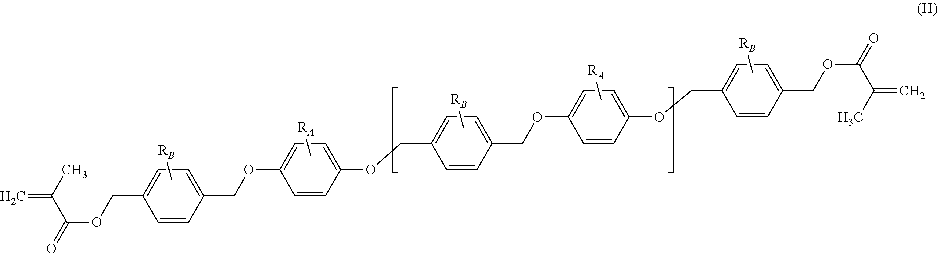

(In Formula (H), R_{A} and R_{B} are a substituent shown in Table 9. Me in Table 9 represents a methyl group. n is a numerical value shown in Table 6 and Table 7.)

TABLE 9

| | Substituent R_{A} | Substituent R_{B} |
|---|---|---|
| Synthesis Example 20 | H | H |
| Synthesis Example 21 | H | Me |
| Synthesis Example 22 | Me | Me |
| Synthesis Example 27 | CF₃ | H |
| Synthesis Example 28 | F | H |
| Synthesis Example 29 | Cl | H |
| Synthesis Example 30 | Br | H |
| Synthesis Example 31 | NO₂ | H |
| Synthesis Example 32 | CF₃ | Me |
| Synthesis Example 33 | F | Me |
| Synthesis Example 34 | Cl | Me |
| Synthesis Example 35 | Br | Me |
| Synthesis Example 36 | NO₂ | Me |
| Synthesis Example 37 | Me | F |
| Synthesis Example 38 | Me | Cl |
| Synthesis Example 39 | Me | Br |
| Synthesis Example 40 | Me | NO₂ |
| Synthesis Example 41 | F | F |
| Synthesis Example 42 | F | Cl |

The compounds of Synthesis Example 23 to Synthesis Example 25 and Synthesis Example 43 to Synthesis Example 51 are indicated by General Formula (I).

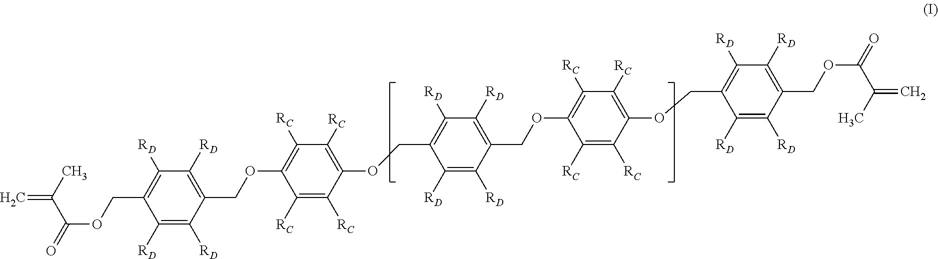

(In Formula (I), R_{C} and R_{D} are a substituent shown in Table 10. Me in Table 10 represents a methyl group. n is a numerical value shown in Table 6 or Table 7.)

TABLE 10

| | Substituent R_{A} | Substituent R_{B} |
|---|---|---|
| Synthesis Example 23 | Me | H |
| Synthesis Example 24 | H | Me |
| Synthesis Example 25 | Me | Me |
| Synthesis Example 43 | F | H |
| Synthesis Example 44 | Cl | H |
| Synthesis Example 45 | Br | H |
| Synthesis Example 46 | H | F |
| Synthesis Example 47 | H | Cl |
| Synthesis Example 48 | H | Br |
| Synthesis Example 49 | F | F |
| Synthesis Example 50 | Cl | Cl |
| Synthesis Example 51 | Br | Br |

The compound of Synthesis Example 26 is indicated by General Formula (J).

The compound of Synthesis Example 52 is indicated by General Formula (K).

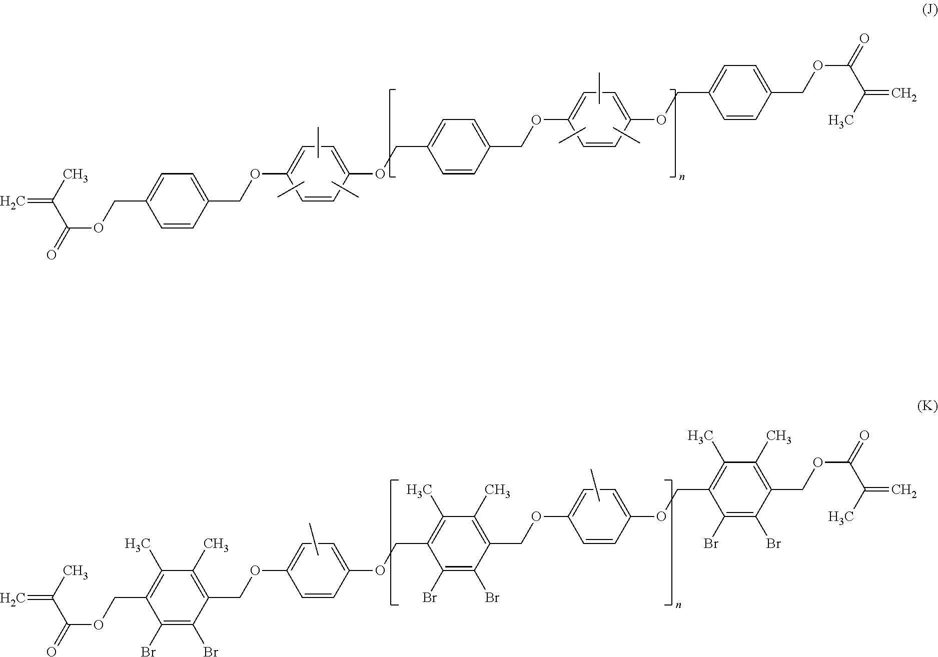

(In Formula (J), n is a numerical value shown in Table 6.)

(In Formula (K), n is a numerical value shown in Table 8.)

The compound of Synthesis Example 53 is indicated by General Formula (L).

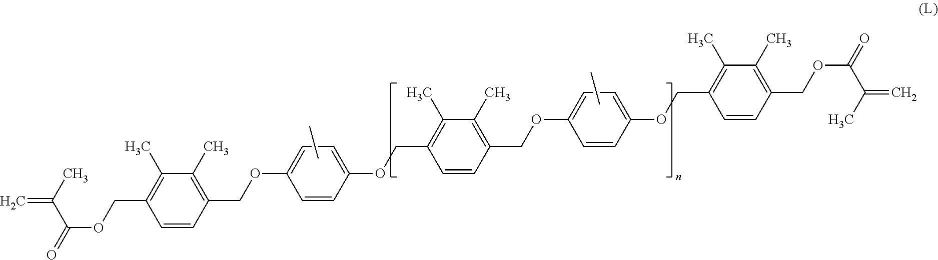

(In Formula (L), n is a numerical value shown in Table 8.)

The compounds of Synthesis Example 54 to Synthesis Example 67 are indicated by General Formula (2) shown above (in Formula (2), Ar₁ an Ar₂ are each an aromatic cyclic group shown in Table 11 or Table 12, and Ar₃ is identical to Ar₂. Z is an end group having a reactive group that is indicated by Formula (M) below. n is a numerical value shown in Table 8.).

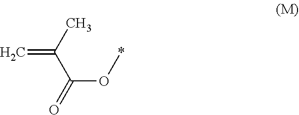

TABLE 11

| | Ar₁ | Ar₂ |
|---|---|---|
| SYNTHESIS EXAMPLE 54 | 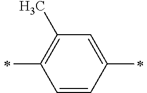 | 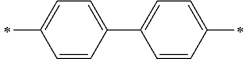 |
| SYNTHESIS EXAMPLE 55 | 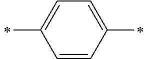 | 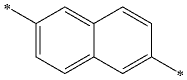 |
| SYNTHESIS EXAMPLE 56 | 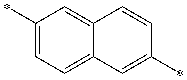 | 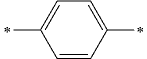 |
| SYNTHESIS EXAMPLE 57 | 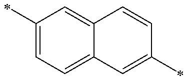 | 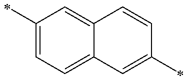 |
| SYNTHESIS EXAMPLE 58 | 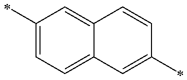 | 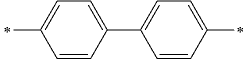 |
| SYNTHESIS EXAMPLE 59 | 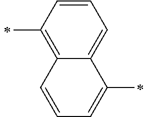 | 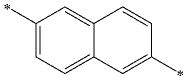 |

TABLE 12

| | Ar₁ | Ar₂ |
|---|---|---|
| SYNTHESIS EXAMPLE 60 | 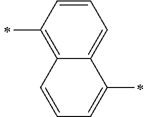 | 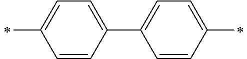 |
| SYNTHESIS EXAMPLE 61 | 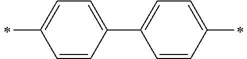 | 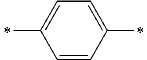 |
| SYNTHESIS EXAMPLE 62 | 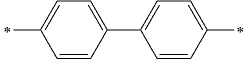 | 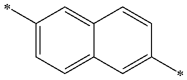 |
| SYNTHESIS EXAMPLE 63 | 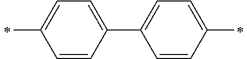 | 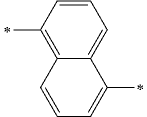 |
| SYNTHESIS EXAMPLE 64 | 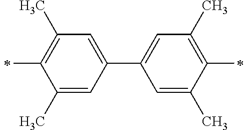 | 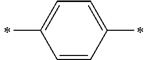 |
| SYNTHESIS EXAMPLE 65 | 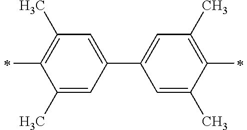 | 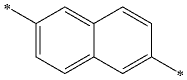 |
| SYNTHESIS EXAMPLE 66 | 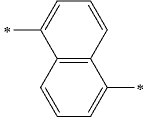 | 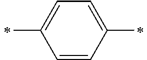 |
| SYNTHESIS EXAMPLE 67 | 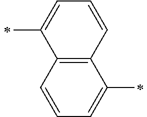 | 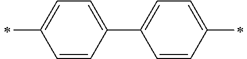 |

The compounds of Synthesis Example 68, Synthesis Example 70 and Synthesis Example 71 are indicated by General Formula (N).

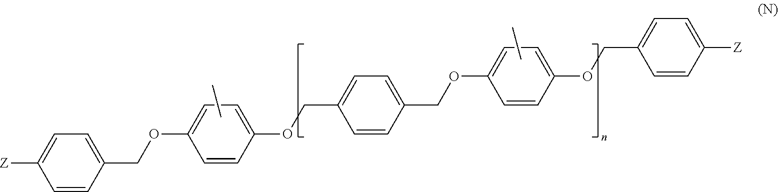

(In Formula (N), Z is an end group having a reactive group shown in Table 13. n is a numerical value shown in Table 8.)

TABLE 13

| | Z |
|---|---|
| SYNTHESIS EXAMPLE 68 | 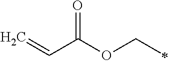 |
| SYNTHESIS EXAMPLE 70 | 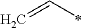 |
| SYNTHESIS EXAMPLE 71 | 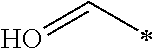 |

The compound of Synthesis Example 69 is indicated by General Formula (C) shown above (in Formula (C), n is a numerical value shown in Table 8.).

As a result of identifying the compounds of Synthesis Example 1 to Synthesis Example 71, as described above, the compounds of Synthesis Example 1 to Synthesis Example 71 were compounds including the first aromatic cyclic units each composed of a first aromatic cyclic group and two ether oxygens bonding to the first aromatic cyclic group, the second aromatic cyclic units each composed of a second aromatic cyclic group and two methylene groups bonding to the second aromatic cyclic group and the third aromatic cyclic units each composed of a third aromatic cyclic group and an end group having a reactive group that bonds to the third aromatic cyclic group, in which a skeleton in which the first aromatic cyclic units and the second aromatic cyclic units were alternately disposed was included, and the first aromatic cyclic units were disposed at both ends of the skeleton and bonded to the third aromatic cyclic groups with the methylene groups or the second aromatic cyclic units were disposed at both ends of the skeleton and bonded to the third aromatic cyclic groups with the ether oxygens.

In addition, from the measurement results of the molecular weights, the average polymerization degrees, which are the average value of the numbers of the repeating units, of the compounds of Synthesis Example 1 to Synthesis Example 71 were calculated.

In addition, solutions containing the components (compounds) having different molecular weights, which had been separated with GPC, respectively, were dried, the masses thereof were measured, and the fractions (mol %) of the individual components that were contained in the compounds of Synthesis Example 1 to Synthesis Example 71 were calculated.

Table 14 and Table 15 show the fractions of the individual components (compounds) having different number of the repeating units that were contained in the compounds of Synthesis Example 1 to Synthesis Example 71 (the fractions (mol %) of the individual components having different “numbers n of the repeating units”) and the average polymerization degrees.

TABLE 14

| | | | | | | | | | | | | | | | | | | | | | | Average |
|---|---|---|---|---|---|---|---|---|---|---|---|---|---|---|---|---|---|---|---|---|---|---|
| | Number of repeating units n (mol %) | | | | | | | | | | | | | | | | | | | | | polymerization |
| Compound | | 1 | 2 | 3 | 4 | 5 | 6 | 7 | 8 | 9 | 10 | 11 | 12 | 13 | 14 | 15 | 16 | 17 | 18 | 19 | 20 | degree |
| Synthesis Example 1 | 27 | 30 | 28 | 10 | 5 | | | | | | | | | | | | | | | | | 2.4 |
| Synthesis Example 2 | 5 | 18 | 33 | 23 | 14 | 5 | 2 | | | | | | | | | | | | | | | 3.5 |
| Synthesis Example 3 | | 5 | 8 | 17 | 25 | 21 | 16 | 8 | | | | | | | | | | | | | | 5.3 |
| Synthesis Example 4 | | | 2 | 7 | 11 | 15 | 16 | 17 | 14 | 10 | 8 | | | | | | | | | | | 7.4 |
| Synthesis Example 5 | | | | | | | 11 | 26 | 29 | 21 | 13 | | | | | | | | | | | 9.0 |
| Synthesis Example 6 | | | | | | | | | 1 | 4 | 11 | 13 | 18 | 21 | 16 | 12 | 4 | | | | | 13.5 |
| Synthesis Example 7 | | | | | | | | | | | | | 3 | 7 | 12 | 17 | 21 | 17 | 13 | 8 | 2 | 17.0 |
| Synthesis Example 8 | 85 | 15 | | | | | | | | | | | | | | | | | | | | 1.2 |
| Synthesis Example 9 | 12 | 18 | 24 | 17 | 14 | 9 | 6 | | | | | | | | | | | | | | | 3.5 |
| Synthesis Example 10 | 12 | 18 | 24 | 17 | 14 | 9 | 6 | | | | | | | | | | | | | | | 3.5 |
| Synthesis Example 11 | 6 | 14 | 24 | 30 | 19 | 5 | 2 | | | | | | | | | | | | | | | 3.7 |
| Synthesis Example 12 | 30 | 31 | 26 | 11 | 2 | | | | | | | | | | | | | | | | | 2.2 |
| Synthesis Example 13 | 7 | 19 | 28 | 26 | 15 | 4 | 1 | | | | | | | | | | | | | | | 3.4 |
| Synthesis Example 14 | | 6 | 10 | 17 | 29 | 21 | 11 | 6 | | | | | | | | | | | | | | 5.1 |
| Synthesis Example 15 | | | 3 | 5 | 9 | 13 | 19 | 18 | 13 | 11 | 9 | | | | | | | | | | | 7.6 |
| Synthesis Example 16 | | | | | | | 9 | 18 | 25 | 24 | 17 | 7 | | | | | | | | | | 9.4 |
| Synthesis Example 17 | | | | | | | | | 1 | 3 | 9 | 14 | 19 | 22 | 16 | 10 | 6 | | | | | 13.6 |
| Synthesis Example 18 | | | | | | | | | | | | | 1 | 5 | 9 | 15 | 19 | 18 | 15 | 11 | 7 | 17.6 |
| Synthesis Example 19 | 83 | 17 | | | | | | | | | | | | | | | | | | | | 1.2 |
| Synthesis Example 20 | 11 | 21 | 29 | 20 | 13 | 6 | | | | | | | | | | | | | | | | 3.2 |
| Synthesis Example 21 | 8 | 26 | 33 | 21 | 9 | 3 | | | | | | | | | | | | | | | | 3.1 |
| Synthesis Example 22 | 3 | 16 | 35 | 27 | 19 | | | | | | | | | | | | | | | | | 3.4 |
| Synthesis Example 23 | 7 | 20 | 33 | 23 | 14 | 3 | | | | | | | | | | | | | | | | 3.3 |
| Synthesis Example 24 | 12 | 18 | 34 | 25 | 11 | | | | | | | | | | | | | | | | | 3.1 |
| Synthesis Example 25 | 19 | 27 | 25 | 18 | 9 | 2 | | | | | | | | | | | | | | | | 2.8 |
| Synthesis Example 26 | 9 | 21 | 32 | 22 | 13 | 3 | | | | | | | | | | | | | | | | 3.2 |
| Synthesis Example 27 | 10 | 18 | 26 | 22 | 17 | 7 | | | | | | | | | | | | | | | | 3.4 |
| Synthesis Example 28 | 17 | 22 | 28 | 18 | 12 | 3 | | | | | | | | | | | | | | | | 3.0 |
| Synthesis Example 29 | 14 | 19 | 27 | 20 | 11 | 9 | | | | | | | | | | | | | | | | 3.2 |
| Synthesis Example 30 | 14 | 20 | 29 | 24 | 12 | 1 | | | | | | | | | | | | | | | | 3.0 |
| Synthesis Example 31 | 18 | 22 | 25 | 23 | 11 | 1 | | | | | | | | | | | | | | | | 2.9 |
| Synthesis Example 32 | 14 | 18 | 26 | 23 | 12 | 7 | | | | | | | | | | | | | | | | 3.2 |
| Synthesis Example 33 | 17 | 24 | 28 | 16 | 9 | 6 | | | | | | | | | | | | | | | | 2.9 |
| Synthesis Example 34 | 13 | 19 | 27 | 21 | 14 | 6 | | | | | | | | | | | | | | | | 3.2 |
| Synthesis Example 35 | 4 | 15 | 25 | 29 | 18 | 9 | | | | | | | | | | | | | | | | 3.7 |

TABLE 15

| | | | | | | | | | | | | | | | | | | | | | | Average |
|---|---|---|---|---|---|---|---|---|---|---|---|---|---|---|---|---|---|---|---|---|---|---|
| | Number of repeating units n (mol %) | | | | | | | | | | | | | | | | | | | | | polymerization |
| Compound | | 1 | 2 | 3 | 4 | 5 | 6 | 7 | 8 | 9 | 10 | 11 | 12 | 13 | 14 | 15 | 16 | 17 | 18 | 19 | 20 | degree |
| Synthesis Example 36 | 10 | 19 | 26 | 22 | 14 | 9 | | | | | | | | | | | | | | | | 3.4 |
| Synthesis Example 37 | 14 | 22 | 27 | 19 | 11 | 7 | | | | | | | | | | | | | | | | 3.1 |
| Synthesis Example 38 | 3 | 15 | 27 | 28 | 18 | 9 | | | | | | | | | | | | | | | | 3.7 |
| Synthesis Example 39 | 7 | 16 | 29 | 21 | 17 | 10 | | | | | | | | | | | | | | | | 3.6 |
| Synthesis Example 40 | 8 | 15 | 27 | 23 | 17 | 10 | | | | | | | | | | | | | | | | 3.6 |
| Synthesis Example 41 | 10 | 19 | 28 | 25 | 17 | 1 | | | | | | | | | | | | | | | | 3.2 |
| Synthesis Example 42 | 10 | 17 | 25 | 22 | 18 | 8 | | | | | | | | | | | | | | | | 3.5 |
| Synthesis Example 43 | 3 | 16 | 29 | 26 | 19 | 7 | | | | | | | | | | | | | | | | 3.6 |
| Synthesis Example 44 | 10 | 21 | 31 | 25 | 10 | 3 | | | | | | | | | | | | | | | | 3.1 |
| Synthesis Example 45 | 14 | 19 | 27 | 23 | 11 | 6 | | | | | | | | | | | | | | | | 3.2 |
| Synthesis Example 46 | 3 | 17 | 32 | 29 | 12 | 7 | | | | | | | | | | | | | | | | 3.5 |
| Synthesis Example 47 | 17 | 31 | 22 | 15 | 11 | 4 | | | | | | | | | | | | | | | | 2.8 |
| Synthesis Example 48 | 2 | 18 | 29 | 25 | 21 | 5 | | | | | | | | | | | | | | | | 3.6 |
| Synthesis Example 49 | 12 | 21 | 30 | 23 | 13 | 1 | | | | | | | | | | | | | | | | 3.1 |
| Synthesis Example 50 | 13 | 18 | 29 | 25 | 9 | 6 | | | | | | | | | | | | | | | | 3.2 |
| Synthesis Example 51 | 4 | 17 | 27 | 26 | 18 | 8 | | | | | | | | | | | | | | | | 3.6 |
| Synthesis Example 52 | 9 | 19 | 26 | 22 | 17 | 7 | | | | | | | | | | | | | | | | 3.4 |
| Synthesis Example 53 | 19 | 25 | 22 | 17 | 11 | 6 | | | | | | | | | | | | | | | | 2.9 |
| Synthesis Example 54 | 11 | 21 | 29 | 20 | 13 | 6 | | | | | | | | | | | | | | | | 3.2 |
| Synthesis Example 55 | 20 | 25 | 22 | 18 | 11 | 4 | | | | | | | | | | | | | | | | 2.9 |
| Synthesis Example 56 | 18 | 23 | 25 | 19 | 13 | 2 | | | | | | | | | | | | | | | | 2.9 |
| Synthesis Example 57 | 21 | 26 | 24 | 17 | 9 | 3 | | | | | | | | | | | | | | | | 2.8 |
| Synthesis Example 58 | 14 | 21 | 25 | 23 | 10 | 7 | | | | | | | | | | | | | | | | 3.2 |
| Synthesis Example 59 | 12 | 19 | 24 | 20 | 15 | 10 | | | | | | | | | | | | | | | | 3.4 |
| Synthesis Example 60 | 24 | 27 | 21 | 14 | 9 | 5 | | | | | | | | | | | | | | | | 2.7 |
| Synthesis Example 61 | 24 | 29 | 25 | 17 | 5 | | | | | | | | | | | | | | | | | 2.5 |
| Synthesis Example 62 | 13 | 22 | 26 | 20 | 14 | 5 | | | | | | | | | | | | | | | | 3.2 |
| Synthesis Example 63 | 19 | 24 | 22 | 17 | 11 | 7 | | | | | | | | | | | | | | | | 3.0 |
| Synthesis Example 64 | 17 | 23 | 25 | 18 | 10 | 7 | | | | | | | | | | | | | | | | 3.0 |
| Synthesis Example 65 | 13 | 20 | 26 | 19 | 16 | 6 | | | | | | | | | | | | | | | | 3.2 |
| Synthesis Example 66 | 20 | 26 | 24 | 17 | 6 | 4 | | | | | | | | | | | | | | | | 2.9 |
| Synthesis Example 67 | 18 | 23 | 25 | 20 | 10 | 4 | | | | | | | | | | | | | | | | 2.9 |
| Synthesis Example 68 | 7 | 19 | 28 | 26 | 15 | 4 | 1 | | | | | | | | | | | | | | | 3.4 |
| Synthesis Example 69 | 5 | 18 | 33 | 23 | 14 | 5 | 2 | | | | | | | | | | | | | | | 3.5 |
| Synthesis Example 70 | 7 | 19 | 28 | 26 | 15 | 4 | 1 | | | | | | | | | | | | | | | 3.4 |
| Synthesis Example 71 | 7 | 19 | 28 | 26 | 15 | 4 | 1 | | | | | | | | | | | | | | | 3.4 |

### Production of Polymerization Products

### Examples 1 to 8

A compound shown in Table 16 and terephthalic acid, which was a monomer as a different component 1, were mixed together in fractions shown in Table 16, respectively, and reacted in a vacuum at 250° C. to be polymerized, thereby obtaining a polymerization product (polyester) of each of Examples 1 to 8.

### Example 9

As a compound, a compound obtained by mixing the compound of Synthesis Example 7 and the compound of Synthesis Example 8 in fractions of 1:1 in terms of the mass ratio was used, the compound and terephthalic acid as the different component 1 were mixed together in the fractions shown in Table 16, respectively, and reacted in a vacuum at 250° C. to be polymerized, thereby obtaining a polymerization product (polyester) of Example 9.

### Example 10

As a compound, a compound obtained by mixing the compound of Synthesis Example 5, the compound of Synthesis Example 7 and the compound of Synthesis Example 8 in fractions of 1:1:1 in terms of the mass ratio was used, the compound and terephthalic acid as the different component 1 were mixed together in the fractions shown in Table 16, respectively, and reacted in a vacuum at 250° C. to be polymerized, thereby obtaining a polymerization product (polyester) of 10.

### Example 11

As a compound, a compound obtained by mixing the compound of Synthesis Example 1, the compound of Synthesis Example 4, the compound of Synthesis Example 6 and the compound of Synthesis Example 7 in fractions of 1:1:1:1 in terms of the mass ratio was used, the compound and terephthalic acid as the different component 1 were mixed together in the fractions shown in Table 16, respectively, and reacted in a vacuum at 250° C. to be polymerized, thereby obtaining a polymerization product (polyester) of Example 11.

### Examples 12 to 14

4-Acetoxybenzoic acid as a different component 2 was mixed into a resin composition which was obtained by mixing a compound shown in Table 16 and terephthalic acid, which was a monomer as the different component 1, in fractions shown in Table 16 in the same manner as in Example 2 in fractions shown in Table 16, and reacted in a vacuum at 250° C. to be polymerized, thereby obtaining a polymerization product (polyester) of each of Examples 12 to 14.

### Example 15

The compound shown in Table 16 was dissolved in N-methyl-2-pyrrolidone (NMP), triethylamine was added thereto, an NMP solution of p-phenylenediamine was added dropwise thereto at 0° C., the solution was stirred at 0° C. for four hours, then, heated up to 100° C. and stirred for four hours, thereby causing a reaction and polymerization. The obtained suspension was poured into water and stirred for 30 minutes, and the generated precipitate was filtered and recovered. The recovered precipitate was heated and dried at 100° C. for 24 hours in a vacuum, thereby obtaining a polymerization product (polyamide) of Example 15.

### Example 16

Terephthaloyl dichloride was dissolved in N,N-dimethylformamide (DMF) in a three-neck flask in a nitrogen atmosphere, and triethylamine was added thereto, thereby producing a mixed solution. Next, a solution obtained by dissolving the compound of Synthesis Example 11 in DME was added dropwise to the mixed solution that had been cooled to 0° C., stirred at 0° C. for four hours, then, heated up to 100° C. and stirred for four hours, thereby causing a reaction and polymerization. After the end of the reaction, the obtained suspension was poured into water and stirred for 30 minutes, and the generated precipitate was filtered and recovered. The recovered precipitate was heated and dried at 100° C. for 24 hours in a vacuum, thereby obtaining a polymerization product (polyester) of Example 16.

TABLE 16

| | | | | | | | | Deflection | |
|---|---|---|---|---|---|---|---|---|---|
| | | | | | Different | Different | Thermal | tempera- | Processing |
| Resin | | | | | component | component | conduc- | ture under | tempera- |
| cured | | Different | Different | Compound | 1 | 2 | tivity | load | ture |
| product | Compound | component 1 | component 2 | (mass %) | (mass %) | (mass %) | W/(m · K) | (° C.) | (° C.) |
| Example 1 | Synthesis Example 1 | Terephthalic acid | | 80 | 20 | | 0.6 | 185 | 230 |
| Example 2 | Synthesis Example 2 | Terephthalic acid | | 85 | 15 | | 0.7 | 191 | 240 |
| Example 3 | Synthesis Example 3 | Terephthalic acid | | 90 | 10 | | 0.6 | 206 | 250 |
| Example 4 | Synthesis Example 4 | Terephthalic acid | | 93 | 7 | | 0.7 | 211 | 250 |
| Example 5 | Synthesis Example 5 | Terephthalic acid | | 95 | 5 | | 0.7 | 231 | 280 |
| Example 6 | Synthesis Example 6 | Terephthalic acid | | 97 | 3 | | 0.6 | 240 | 300 |
| Example 7 | Synthesis Example 7 | Terephthalic acid | | 98 | 2 | | 0.6 | 267 | 300 |
| Example 8 | Synthesis Example 8 | Terephthalic acid | | 70 | 30 | | 0.5 | 181 | 230 |
| Example 9 | Synthesis Example 7, | Terephthalic acid | | 85 | 15 | | 0.5 | 216 | 250 |
| | 8 | | | | | | | | |
| Example 10 | Synthesis Example 5, | Terephthalic acid | | 88 | 12 | | 0.5 | 223 | 270 |
| | 7, 8 | | | | | | | | |
| Example 11 | Synthesis Example 1, | Terephthalic acid | | 93 | 7 | | 0.7 | 231 | 280 |
| | 4, 6, 7 | | | | | | | | |
| Example 12 | | Terephthalic acid | 4-Acetoxy- | 0 | 0 | 10 | 0.8 | 281 | 330 |
| | | | benzoic acid | | | | | | |
| Example 13 | | Terephthalic acid | 4-Acetoxy- | 0 | 0 | 50 | 0.6 | 318 | 360 |
| | | | benzoic acid | | | | | | |
| Example 14 | | Terephthalic acid | 4-Acetoxy- | 0 | 0 | 90 | 0.5 | 355 | 400 |
| | | | benzoic acid | | | | | | |
| Example 15 | Synthesis Example 10 | p-Phenylene- | | 85 | 15 | | 0.6 | 197 | 250 |
| | | diamine | | | | | | | |
| Example 16 | Synthesis Example 11 | Terephthaloyl | | 84 | 16 | | 0.6 | 206 | 250 |
| | | dichloride | | | | | | | |

For the polymerization products of Examples 1 to 16 obtained as described above, the processing temperature, the deflection temperature under load and the thermal conductivity were obtained, respectively, by methods described below. The results are shown in Table 16.

### (Measurement of Processing Temperature)

Each polymerization product was heated on a hot plate, and a temperature at which softening behaviors began was measured and regarded as the processing temperature.

### (Measurement of Deflection Temperature Under Load)

The deflection temperature under load was measured according to the method of JIS 7191.

### (Measurement of Thermal Conductivity)

The density, specific heat and thermal diffusivity of the polymer (polymerization product) obtained in each of Examples 1 to 16 were measured, respectively, and multiplied by one another, thereby obtaining the thermal conductivity.

The density was obtained using the Archimedes method.

For the specific heat, a specific heat at 25° C. was calculated according to JIS K 7123 using a differential scanning calorimeter (DSC) (manufactured by Hitachi High-Tech Science Corporation).

The thermal diffusivity was obtained using a thermal diffusivity measurement system by the Xe flash method (Advance Riko, Inc.).

A sample for thermal diffusivity measurement was produced by a method described below.

That is, a 1 mm-thick plate-shape sample was produced from each polymerization product by a vacuum heating and pressing method at the processing temperature of each polymerization product and a pressure of 3 MPa, and the sample was processed into a cylindrical shape that was 10 mm in diameter and 1 mm in thickness and used as the sample for thermal diffusivity measurement.

As shown in Table 16, all of the polymerization products of Examples 1 to 16 had a thermal conductivity of 0.5 W/(m·K) or higher, and the polymerization products had a high thermal conductivity.

In addition, all of the polymerization products of Examples 1 to 16 had a high deflection temperature under load and favorable heat resistance.

### Examples 17 to 24, 28 and 37 to 84

A compound shown in Table 17 and Table 18 and PERHEXYL D (trade name: manufactured by NOF Corporation), which is a radical polymerization initiator, as a different component were mixed together in fractions shown in Table 17 and Table 18, heated up to 150° C. in a vacuum and polymerized in a molten state, thereby obtaining a polymerization product (acryl polymerization product) of each of Examples 17 to 24, 28 and 37 to 84.

### Example 25

As a compound, a compound obtained by mixing the compound of Synthesis Example 18 and the compound of Synthesis Example 19 in fractions of 1:1 in terms of the mass ratio was used, PERHEXYL D (trade name: manufactured by NOF Corporation), which is a radical polymerization initiator, was mixed thereinto as a different component in the fraction shown in Table 17, and the compound and the different component were polymerized in the same manner as in Example 17, thereby obtaining a polymerization product (acryl polymerization product) of Example 25.

### Example 26

As a compound, a compound obtained by mixing the compound of Synthesis Example 16, the compound of Synthesis Example 18 and the compound of Synthesis Example 19 in fractions of 1:1:1 in terms of the mass ratio was used, PERHEXYL D (trade name: manufactured by NOF Corporation), which is a radical polymerization initiator, was mixed thereinto as a different component in the fraction shown in Table 17, and the compound and the different component were polymerized in the same manner as in Example 17, thereby obtaining a polymerization product (acryl polymerization product) of Example 26.

### Example 27

As a compound, a compound obtained by mixing the compound of Synthesis Example 12, the compound of Synthesis Example 15, the compound of Synthesis Example 17 and the compound of Synthesis Example 18 in fractions of 1:1:1:1 in terms of the mass ratio was used, PERHEXYL D (trade name: manufactured by NOF Corporation), which is a radical polymerization initiator, was mixed thereinto as a different component in the fraction shown in Table 17, and the compound and the different component were polymerized in the same manner as in Example 17, thereby obtaining a polymerization product (acryl polymerization product) of Example 27.

### Examples 29 to 31

A compound shown in Table 17, YL6121 (manufactured by Mitsubishi Chemical Corporation), which is an epoxy resin, as a different component and 2-ethyl-4-methylimidazole (2E4MZ (manufactured by Shikoku Chemicals Corporation)), which is a polymerization accelerator, were mixed together in fractions shown in Table 17 (in Table 17, the fraction of 2E4MZ is shown in a parenthesis) and polymerized in a molten state at 150° C., thereby obtaining a polymerization product (epoxy polymerization product) of each of Examples 29 to 31.

The epoxy resin (YL6121) used as a material for the resin compositions of Examples 29 to 31 is a mixture of a compound having an epoxy group that is indicated by Formula (15) below and a compound having an epoxy group that is indicated by (16).

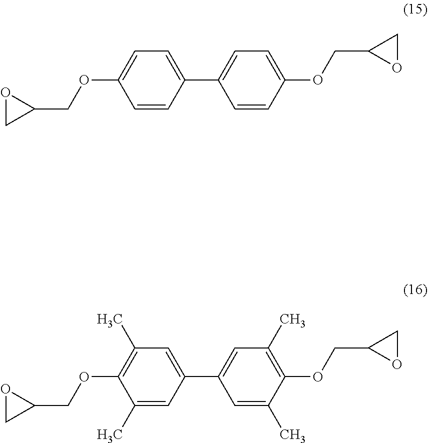 [SMILES: c1cc(-c2ccc(OCC3CO3)cc2)ccc1OCC1CO1 ; Cc1cc(-c2cc(C)c(OCC3CO3)c(C)c2)cc(C)c1OCC1CO1]

### Example 32

A compound shown in Table 17 and 2-ethyl-4-methylimidazole (2E4MZ (manufactured by Shikoku Chemicals Corporation)), which is a polymerization accelerator, as a different component were mixed together in the fractions shown in Table 17 and polymerized in a molten state at 150° C., thereby obtaining a polymerization product (epoxy polymerization product) of Example 32.

### Example 33

A compound shown in Table 17 and cyclohexyl p-toluenesulfonate (manufactured by Tokyo Chemical Industry Co., Ltd.), which is a curing agent, as a different component were mixed together in the fractions shown in Table 17 and polymerized in a molten state at 150° C., thereby obtaining a polymerization product (epoxy polymerization product) of Example 33.

### Example 34

The compound shown in Table 17 and PERHEXYL D (trade name: manufactured by NOF Corporation), which is a radical polymerization initiator, as a different component were mixed together in the fractions shown in Table 17 and polymerized in a molten state at 150° C., thereby obtaining a polymerization product of Examples 34.

### Example 35

Pyromellitic dianhydride was dissolved in N-methyl-2-pyrrolidone (NMP) in a three-neck flask in a nitrogen atmosphere, a solution obtained by dissolving the compound of Synthesis Example 11 in the NMP was applied with an applicator with a 200 μm gap, the coated film was dried at 80° C. and then polymerized at 150° C. for three hours, 200° C. for three hours and 300° C. for three hours, thereby obtaining a polymerization product (polyimide) of Example 35.

### Example 36

The compound shown in Table 17 and 4,4′-diisocyanato-3,3′-dimethylbiphenyl as a different component were mixed together in the fractions shown in Table 17 and polymerized in a molten state at 150° C., thereby obtaining a polymerization product of Examples 36.

TABLE 17

| | | | | Different | Thermal |
|---|---|---|---|---|---|
| Resin cured | | | Compound | component | conductivity |
| product | Compound | Different component | (mass %) | (mass %) | W/(m · K) |
| Example 17 | Synthesis Example 12 | PERHEXYL D | 99 | 1 | 0.5 |
| Example 18 | Synthesis Example 13 | PERHEXYL D | 99 | 1 | 0.7 |
| Example 19 | Synthesis Example 14 | PERHEXYL D | 99 | 1 | 0.8 |
| Example 20 | Synthesis Example 15 | PERHEXYL D | 99 | 1 | 0.7 |
| Example 21 | Synthesis Example 16 | PERHEXYL D | 99 | 1 | 0.6 |
| Example 22 | Synthesis Example 17 | PERHEXYL D | 99 | 1 | 0.5 |
| Example 23 | Synthesis Example 18 | PERHEXYL D | 99 | 1 | 0.5 |
| Example 24 | Synthesis Example 19 | PERHEXYL D | 99 | 1 | 0.5 |
| Example 25 | Synthesis Example 18, 19 | PERHEXYL D | 99 | 1 | 0.5 |
| Example 26 | Synthesis Example 16, 18, 19 | PERHEXYL D | 99 | 1 | 0.5 |
| Example 27 | Synthesis Example 12, 15, 17, | PERHEXYL D | 99 | 1 | 0.6 |
| | 18 | | | | |
| Example 28 | Synthesis Example 66 | PERHEXYL D | 99 | 1 | 0.7 |
| Example 29 | Synthesis Example 67 | Epoxy resin YL6121-2E4MZ | 70 | 15 (15) | 0.6 |
| Example 30 | Synthesis Example 9 | Epoxy resin YL6121-2E4MZ | 63 | 22 (15) | 0.6 |
| Example 31 | Synthesis Example 11 | Epoxy resin YL6121-2E4MZ | 35 | 50 (15) | 0.7 |
| Example 32 | Synthesis Example 67 | 2E4MZ | 85 | 15 | 0.6 |
| Example 33 | Synthesis Example 67 | Cyclohexyl p-toluenesulfonate | 85 | 15 | 0.6 |
| Example 34 | Synthesis Example 68 | PERHEXYL D | 99 | 1 | 0.7 |
| Example 35 | Synthesis Example 11 | Pyromellitic dianhydride | 85 | 15 | 0.5 |
| Example 36 | Synthesis Example 69 | 4,4′-Diisocyanato-3,3′- | 85 | 15 | 0.5 |
| | | dimethylbiphenyl | | | |
| Example 37 | Synthesis Example 20 | PERHEXYL D | 99 | 1 | 0.6 |
| Example 38 | Synthesis Example 21 | PERHEXYL D | 99 | 1 | 0.8 |
| Example 39 | Synthesis Example 22 | PERHEXYL D | 99 | 1 | 0.7 |
| Example 40 | Synthesis Example 23 | PERHEXYL D | 99 | 1 | 0.6 |
| Example 41 | Synthesis Example 24 | PERHEXYL D | 99 | 1 | 0.6 |
| Example 42 | Synthesis Example 25 | PERHEXYL D | 99 | 1 | 0.7 |
| Example 43 | Synthesis Example 26 | PERHEXYL D | 99 | 1 | 0.7 |
| Example 44 | Synthesis Example 27 | PERHEXYL D | 99 | 1 | 0.6 |
| Example 45 | Synthesis Example 28 | PERHEXYL D | 99 | 1 | 0.7 |
| Example 46 | Synthesis Example 29 | PERHEXYL D | 99 | 1 | 0.6 |
| Example 47 | Synthesis Example 30 | PERHEXYL D | 99 | 1 | 0.5 |
| Example 48 | Synthesis Example 31 | PERHEXYL D | 99 | 1 | 0.5 |
| Example 49 | Synthesis Example 32 | PERHEXYL D | 99 | 1 | 0.7 |
| Example 50 | Synthesis Example 33 | PERHEXYL D | 99 | 1 | 0.7 |

TABLE 18

| | | | | Different | Thermal |
|---|---|---|---|---|---|
| Resin cured | | | Compound | component | conductivity |
| product | Compound | Different component | (mass %) | (mass %) | W/(m · K) |
| Example 51 | Synthesis Example 34 | PERHEXYL D | 99 | 1 | 0.6 |
| Example 52 | Synthesis Example 35 | PERHEXYL D | 99 | 1 | 0.5 |
| Example 53 | Synthesis Example 36 | PERHEXYL D | 99 | 1 | 0.5 |
| Example 54 | Synthesis Example 37 | PERHEXYL D | 99 | 1 | 0.7 |
| Example 55 | Synthesis Example 38 | PERHEXYL D | 99 | 1 | 0.6 |
| Example 56 | Synthesis Example 39 | PERHEXYL D | 99 | 1 | 0.5 |
| Example 57 | Synthesis Example 40 | PERHEXYL D | 99 | 1 | 0.5 |
| Example 58 | Synthesis Example 41 | PERHEXYL D | 99 | 1 | 0.7 |
| Example 59 | Synthesis Example 42 | PERHEXYL D | 99 | 1 | 0.6 |
| Example 60 | Synthesis Example 43 | PERHEXYL D | 99 | 1 | 0.7 |
| Example 61 | Synthesis Example 44 | PERHEXYL D | 99 | 1 | 0.6 |
| Example 62 | Synthesis Example 45 | PERHEXYL D | 99 | 1 | 0.5 |
| Example 63 | Synthesis Example 46 | PERHEXYL D | 99 | 1 | 0.7 |
| Example 64 | Synthesis Example 47 | PERHEXYL D | 99 | 1 | 0.6 |
| Example 65 | Synthesis Example 48 | PERHEXYL D | 99 | 1 | 0.5 |
| Example 66 | Synthesis Example 49 | PERHEXYL D | 99 | 1 | 0.7 |
| Example 67 | Synthesis Example 50 | PERHEXYL D | 99 | 1 | 0.5 |
| Example 68 | Synthesis Example 51 | PERHEXYL D | 99 | 1 | 0.5 |
| Example 69 | Synthesis Example 52 | PERHEXYL D | 99 | 1 | 0.5 |
| Example 70 | Synthesis Example 53 | PERHEXYL D | 99 | 1 | 0.5 |
| Example 71 | Synthesis Example 54 | PERHEXYL D | 99 | 1 | 0.5 |
| Example 72 | Synthesis Example 55 | PERHEXYL D | 99 | 1 | 0.5 |
| Example 73 | Synthesis Example 56 | PERHEXYL D | 99 | 1 | 0.5 |
| Example 74 | Synthesis Example 57 | PERHEXYL D | 99 | 1 | 0.6 |
| Example 75 | Synthesis Example 58 | PERHEXYL D | 99 | 1 | 0.6 |
| Example 76 | Synthesis Example 59 | PERHEXYL D | 99 | 1 | 0.5 |
| Example 77 | Synthesis Example 60 | PERHEXYL D | 99 | 1 | 0.7 |
| Example 78 | Synthesis Example 61 | PERHEXYL D | 99 | 1 | 0.6 |
| Example 79 | Synthesis Example 62 | PERHEXYL D | 99 | 1 | 0.7 |
| Example 80 | Synthesis Example 63 | PERHEXYL D | 99 | 1 | 0.5 |
| Example 81 | Synthesis Example 64 | PERHEXYL D | 99 | 1 | 0.6 |
| Example 82 | Synthesis Example 65 | PERHEXYL D | 99 | 1 | 0.7 |
| Example 83 | Synthesis Example 66 | PERHEXYL D | 99 | 1 | 0.5 |
| Example 84 | Synthesis Example 67 | PERHEXYL D | 99 | 1 | 0.5 |

For each of the polymerization products of Examples 17 to 84 obtained as described above, the thermal conductivity was obtained by a method described below. The results are shown in Table 17 and Table 18.

### (Measurement of Thermal Conductivity)

The density, specific heat and thermal diffusivity of the polymer (polymerization product) obtained in each of the polymerization products of Examples 17 to 84 were measured, respectively, and multiplied by one another, thereby obtaining the thermal conductivity.

The density was obtained using the Archimedes method.

For the specific heat, a specific heat at 25° C. was calculated according to JIS K 7123 using a differential scanning calorimeter (DSC) (manufactured by Hitachi High-Tech Science Corporation).

The thermal diffusivity was obtained using a thermal diffusivity measurement system by the Xe flash method (Advance Riko, Inc.).

A sample for thermal diffusivity measurement was processed into a cylindrical shape that was 10 mm in diameter and 1 mm in thickness.

In Examples 17 to 34 and 36 to 84, the samples for measurement were produced by a method in which the resin composition to be polymerized was polymerized in a heated and molten state at 150° C. in an aluminum formwork.

In Example 35, the sample for measurement was produced by a method described below. That is, the resin composition to be polymerized was applied onto a mold release agent-applied aluminum foil using a 20 μm applicator and dried at 80° C. After that, the resin composition was heated at 150° C. for three hours and, furthermore, at 200° C. for three hours and polymerized, thereby producing a polymerized film. The resin composition was applied, dried and polymerized a plurality of times on the obtained polymerized film such that the thickness of the polymerized film reached 1 mm to produce a 1 mm-thick plate-shaped sample, and the sample was processed into a cylindrical shape that is 10 mm in diameter and 1 mm in thickness and used as a sample for thermal diffusivity measurement.

As shown in Table 17 and Table 18, all of the polymerization products of Examples 17 to 82 had a thermal conductivity of 0.5 W/(m·K) or higher, and the polymerization products had a high thermal conductivity.

### INDUSTRIAL APPLICABILITY

The present disclosure provides a compound from which a polymer having a high thermal conductivity can be obtained.

1. A compound comprising,
end groups each having a reactive group that are disposed at both ends respectively, and
between the end groups, either or both of:
a first structure in which an aromatic cyclic group, an ether oxygen, a methylene group, an aromatic cyclic group, a methylene group, an ether oxygen and an aromatic cyclic group are bonded together in this order, and
a second structure in which an aromatic cyclic group, a methylene group, an ether oxygen, an aromatic cyclic group, an ether oxygen, a methylene group and an aromatic cyclic group are bonded together in this order.

2. The compound according to claim 1, comprising:
a first aromatic cyclic unit composed of a first aromatic cyclic group and two ether oxygens bonding to the first aromatic cyclic group;
a second aromatic cyclic unit composed of a second aromatic cyclic group and two methylene groups bonding to the second aromatic cyclic group; and
a third aromatic cyclic unit composed of a third aromatic cyclic group and an end group having a reactive group that bonds to the third aromatic cyclic group,
wherein the compound comprises a skeleton in which the first aromatic cyclic units and the second aromatic cyclic units are alternately disposed, and
the first aromatic cyclic units are disposed at both ends of the skeleton and bonded to the third aromatic cyclic groups via methylene groups, or the second aromatic cyclic units are disposed at both ends of the skeleton and bonded to the third aromatic cyclic groups via ether oxygens.

3. The compound according to claim 1 that is represented by General Formula (1) below or General Formula (2) below, 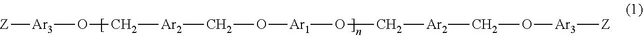
(in Formula (1), Ar₁ each independently represents a first aromatic cyclic group that may have a substituent, Ar₂ each independently represents a second aromatic cyclic group that may have a substituent, and Ar₃ each independently represents a third aromatic cyclic group that may have a substituent; Z each independently represents an end group having a reactive group; and n is an integer of 0 or larger,) 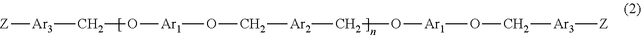
(in Formula (2), Ar₁ each independently represents a first aromatic cyclic group that may have a substituent, Ar₂ each independently represents a second aromatic cyclic group that may have a substituent, and Ar₃ each independently represents a third aromatic cyclic group that may have a substituent; Z each independently represents an end group having a reactive group; and n is an integer of 0 or larger.)

4. The compound according to claim 2,
wherein any one or more of the first aromatic cyclic group, the second aromatic cyclic group and the third aromatic cyclic group are any of aromatic cyclic groups represented by General Formulae (3) to (7) below, 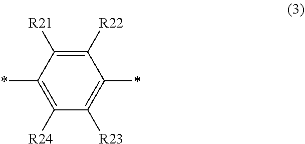
(in Formula (3), R21 to R24 are each independently any one selected from the group consisting of hydrogen, a methyl group, a trifluoromethyl group, a halogen group and a nitro group,) 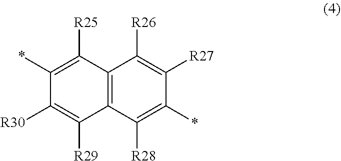
(in Formula (4), R25 to R30 are each independently any one selected from the group consisting of hydrogen, a methyl group, a trifluoromethyl group, a halogen group and a nitro group,) 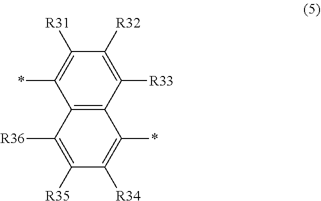
(in Formula (5), R31 to R36 are each independently any one selected from the group consisting of hydrogen, a methyl group, a trifluoromethyl group, a halogen group and a nitro group,) 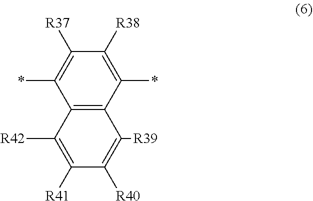
(in Formula (6), R37 to R42 are each independently any one selected from the group consisting of hydrogen, a methyl group, a trifluoromethyl group, a halogen group and a nitro group,) 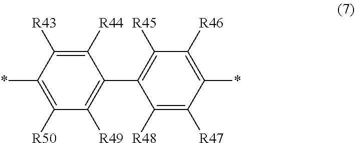
(in Formula (7), R43 to R50 are each independently any one selected from the group consisting of hydrogen, a methyl group, a trifluoromethyl group, a halogen group and a nitro group.)

5. The compound according to claim 2,
wherein any one or more of the first aromatic cyclic group, the second aromatic cyclic group and the third aromatic cyclic group are a para-phenylene group that may have a substituent.

6. The compound according to claim 2,
wherein the second aromatic cyclic group is a para-phenylene group.

7. The compound according to claim 1 that is represented by General Formula (8) below or General Formula (9) below, 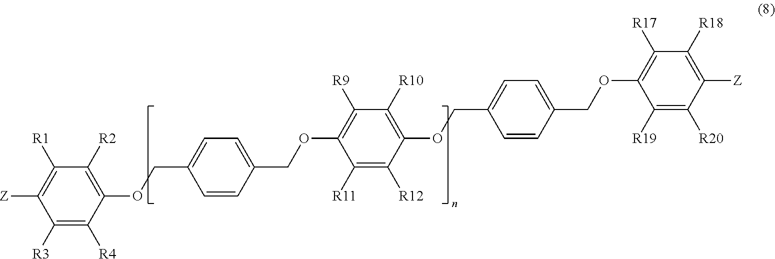
(in Formula (8), R1 to R4, R9 to R12 and R17 to R20 are each independently any one selected from the group consisting of hydrogen, a methyl group, a trifluoromethyl group, a halogen group and a nitro group; Z each independently represents an end group having a reactive group; and n is an integer of 0 or larger,) 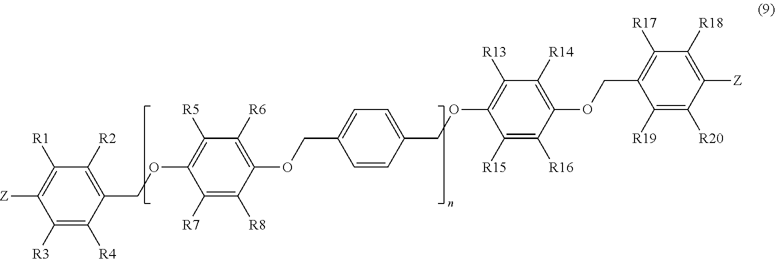
(in Formula (9), R1 to R8 and R13 to R20 are each independently any one selected from the group consisting of hydrogen, a methyl group, a trifluoromethyl group, a halogen group and a nitro group; Z each independently represents an end group having a reactive group; and n is an integer of 0 or larger.)

8. The compound according to claim 1,
wherein the end group having a reactive group is —OH, —COOR (R is an alkyl group), —NH₂, —COOH, —COCl, —CH═CH₂, —CH₂OH, —O—COR (R is an alkyl group) or any of end groups represented by Formulae (10) to (12) below,

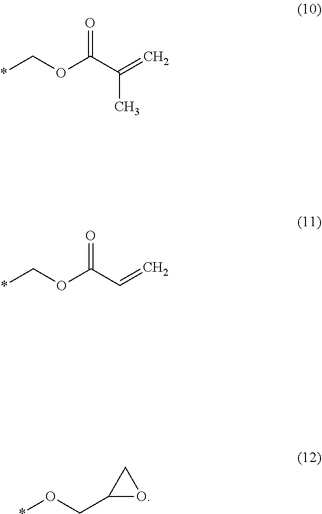

9. A resin composition comprising:
the compound according to claim 1.

10. A polymerization product comprising:
a polymer of the resin composition according to claim 9.
